(19) United States
(12) Patent Application Publication
(10) Pub. No.: US 2026/0345723 A1
(43) Pub. Date: Nov. 5, 2026

(54) Treatments for Opioid-Induced Respiratory Depression

(71) Applicant: Creative Bio-Peptides, Inc., Potomac, MD (US)

(72) Inventor: Michael RUFF, Potomac, MD (US)

(21) Appl. No.: 19/486,605

(22) PCT Filed: May 22, 2024
(86) PCT No.: PCT/US2024/030461

§ 371 Date: Nov. 21, 2025

Related U.S. Application Data
(60) Provisional application No. 63/468,291, filed on May 23, 2023.
(60) Provisional application No. 63/522,191, filed on Jun. 21, 2023.

(51) Int. Cl. A61K 38/08; A61K 9/19; A61K 31/485; A61P 25/36

(52) U.S. Cl. CPC A61K 38/08; A61K 9/19; A61K 31/485; A61P 25/36

(57) ABSTRACT

Disclosed herein are peptides, derivatives thereof, and salts thereof, as well as pharmaceutical compositions containing these, useful alone or in combination with other therapies for treating opioid-induced respiratory depression and other diseases.

### CROSS-REFERENCE TO RELATED APPLICATIONS

This application claims priority under 35 U.S.C. § 119 from U.S. Provisional Application Ser. No. 63/468,291 filed on May 23, 2023, and U.S. Provisional Application Ser. No. 63/522,191 filed on Jun. 21, 2023 the disclosures of which are incorporated herein by reference in their entirety.

### STATEMENT AS TO FEDERALLY SPONSORED RESEARCH

This invention was made with government support under Contract numbers R01 DA045499, P30DA013429, 1R43DA050349, and 2R44DA050349-02 awarded by the National Institute on Drug Abuse. The government has certain rights in the invention.

### SEQUENCE LISTING

The instant application contains a Sequence Listing which has been submitted electronically in ST. 26 xml format and is hereby incorporated by reference in its entirety. Said xml copy, created Apr. 29, 2024, is named 199260-715601_SL.xml and is 54,550 bytes in size.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### SUMMARY

Disclosed herein are methods of treating an opioid-induced respiratory depression in a subject in need thereof. In some embodiments, the method can comprise administering to the subject a therapeutically effective amount of a pharmaceutical composition to treat the opioid-induced respiratory depression. In some embodiments, the pharmaceutical composition can comprise: a peptide, a derivative thereof, or a salt thereof. In some embodiments, the peptide comprises at least five contiguous amino acids or derivatives thereof comprising the general formula: E-F-G-H-I, wherein: E is D-Ser, D-Thr, D-Asn, D-Glu, D-Arg, D-Ile, or D-Leu, or a derivative of any of these; F is D-Ser, D-Thr, D-Asp, or D-Asn, or a derivative of any of these; G is D-Thr, D-Ser, D-Asn, D-Arg, D-Gln, D-Lys, or D-Trp, or a derivative of any of these; H is D-Tyr, or a derivative thereof; and I is D-Thr, D-Ser, D-Arg, or Gly, or a derivative of any of these or a DAPTA, a salt thereof, or a derivative thereof and an excipient, a diluent, a carrier, or a combination thereof. In some embodiments, the administration of the pharmaceutical composition treats the opioid-induced respiratory depression. In some embodiments, the peptide, the derivative thereof, or the salt thereof comprises at least eight contiguous amino acids or derivatives thereof, comprising the general formula A-B-C-E-F-G-H-I, and wherein: A is D-Ala, or a derivative thereof; B is D-Ser, or D-Thr, or a derivative of any of these; C is D-Ser, or D-Thr, or a derivative of any of these; E is D-Ser, D-Thr, D-Asn, D-Glu, D-Arg, D-Ile, or D-Leu, or a derivative of any of these; F is D-Ser, D-Thr, D-Asp, or D-Asn, or a derivative of any of these; G is D-Thr, D-Ser, D-Asn, D-Arg, D-Gln, D-Lys, or D-Trp, or a derivative of any of these; His D-Tyr, or a derivative thereof; and I is D-Thr, D-Ser, D-Arg, or Gly or a derivative of any of these. In some embodiments, the peptide, the derivative thereof, or the salt thereof can be D-Thr, D-Thr, D-Asn, D-Tyr, and D-Thr or a salt thereof. In some embodiments, the derivative of I can be esterified, glycosylated, or amidated at the C terminus. In some embodiments, the peptide can comprise DAPTA, the salt thereof, or the derivative thereof. In some embodiments, the pharmaceutical composition can be in unit dose form. In some embodiments, the pharmaceutical composition can further comprises an excipient, a diluent, a carrier, or a combination thereof. In some embodiments, prior to the administering the subject can be administered an opioid or a salt thereof. In some embodiments, after or concurrently with the administering the subject can be administered an opioid or a salt thereof. In some embodiments, after the administering the subject can have an increased oxygen saturation level as compared to a subject in need thereof who did not receive the pharmaceutical composition. In some embodiments, the oxygen saturation level can be at least 4%, 5%, 6%, 7%, 8%, 9%, or 10% higher in the subject after the administering as compared to the subject in need thereof who did not receive the pharmaceutical composition as measured by mean percent oxygen saturation. In some embodiments, after the administering the subject can have a reduced respiratory rate as compared to a subject in need thereof who did not receive the pharmaceutical composition. In some embodiments, the respiratory rate can be at least 10%, 15%, 20% or 25% lower in the subject after the administering as compared to the subject in need thereof who did not receive the pharmaceutical composition as measured by cumulative respiratory rate. In some embodiments, the administering can be daily, weekly, or monthly. In some embodiments, administering can be once, twice, three, or four times per day. In some embodiments, the pharmaceutical composition can be administered for about: one day, two days, three days, four days, five days, six days, one week, two weeks, three weeks, four weeks, five weeks, one month, two months, three months, four months, five months, six months, seven months, eight months, nine months, ten months, eleven months, one year, two years, or for life. In some embodiments, the pharmaceutical composition can comprise the peptide, the derivative thereof, or the salt thereof in an amount of from about 0.005 mg to about 1000 mg. In some embodiments, the pharmaceutical composition can be administered by: an oral route, an injection route, a sublingual route, a buccal route, a rectal route, a vaginal route, an ocular route, an otic route, a nasal route, an internasal route, an inhalation route, a cutaneous route, a subcutaneous route, an intramuscular route, an intravenous route, a systemic route, a local route, a transdermal route, or any combination thereof. In some embodiments, the pharmaceutical composition can be formulated for oral administration. In some embodiments, the pharmaceutical composition can be in a form of a pill or a liquid. In some embodiments, a second therapy can be administered concurrently or consecutively. In some embodiments, the second therapy can comprise a naloxone, an opioid, a salt of any of these, or any combination thereof. In some embodiments, the pharmaceutical composition can further comprise the second therapy. In some embodiments, the subject can be diagnosed with an opioid use disorder prior to the administering. In some embodiments, the subject can be diagnosed with the opioid-induced respiratory depression prior to the administering. In some embodiments, the diagnoses can comprise an in vitro test, a physical exam, an imaging diagnostic or a combination thereof. In some embodiments, the subject can be a mammal. In some embodiments, the mammal can be a human.

### BRIEF DESCRIPTION OF THE DRAWINGS

FIGS. 1A-1B show RAP-103 prevented the deficit in oxygen saturation caused by acute morphine (MOR) exposure. Rats pretreated with RAP-103 (1 or 3 mg/kg, IP) or a vehicle were treated 30 min later with morphine (5 mg/kg, SC) or saline (SAL), and SpO2 was measured for 60 min. FIG. 1A shows time-course data are expressed as mean % oxygen saturation+S.E.M. N=6-7/group. FIG. 1B shows RAP-103 or vehicle pretreatment expressed across x-axis, and MOR (morphine) or SAL (saline) treatment expressed in legend. Data are expressed as cumulative oxygen saturation for the entire 60 min following morphine treatment (+S.E.M.). ***P<0.001 versus drug-naïve rats (vehicle+SAL) and +++P<0.001 versus morphine-treated rats (vehicle+MOR).

FIGS. 2A-2B show RAP-103 prevented the increase in respiratory rate caused by acute morphine (MOR) exposure. Rats pretreated with RAP-103 (1 or 3 mg/kg, IP) or a vehicle were treated 30 min later with morphine (5 mg/kg, SC) or saline (SAL), and respiratory rate was measured for 60 min. FIG. 2A shows time-course data expressed as mean respiratory rate+S.E.M. N=6-7/group. FIG. 2B shows RAP-103 or vehicle pretreatment expressed across x-axis, and MOR or SAL treatment expressed in legend. Data are expressed as cumulative respiratory rate for the entire 60 min following morphine treatment (+S.E.M.). **P<0.01 versus drug-naïve rats (vehicle+SAL) and ++P<0.01 versus morphine-treated rats (vehicle+MOR).

FIGS. 3A-3B show RAP-103 reduced the severity of morphine physical dependence. Rats were implanted with 75 mg morphine pellets (SC, intact for 7 d) and received daily injections of vehicle or RAP-103 (1 mg/kg, IP). On day 7 (test day), naloxone (10 mg/kg, IP) was administered and withdrawal responses (wet dog shakes and eye blinking) were quantified for 60 min. Wet dog shakes are shown in FIG. 3A. Eye blinking is shown in FIG. 3B. Data are expressed as mean number of wet dog shakes or eye blinking+S.E.M. N=8 rats/group. **P<0.01 or *P<0.05 versus RAP-103-naïve (0 mg/kg) rats that received chronic morphine. Insets) Dose-effect curves for RAP-103 (0.001, 0.1 and 1 mg/kg, IP) against naloxone-precipitated wet dog shakes and shown in FIG. 3A and eye blinking are shown in FIG. 3B. Data are expressed as percentage of control response in the respective RAP-103-naïve groups. N=7-8 rats/group.

FIGS. 4A-4B show RAP-103 reduced acquisition of heroin and heroin reinforcing efficacy in self-administration (SA) assays. FIG. 4A shows rats tested with 10 days of heroin intravenous self-administration (SA) on a Fixed ratio (FR) schedule. Rats underwent intravenous heroin SA (0.1 mg/kg/inf) on a FR schedule of reinforcement for at least 10 days. RAP-103 (1 mg/kg, IP) or a vehicle (VEH) was injected 30 min before each daily heroin SA session. Data are expressed as mean infusions per SA session (6.5 h)+S.E.M. N=7-8 rats/group. ***P<0.001 or *P<0.05 compared to heroin self-administering rats treated with VEH. FIG. 4B shows reinforcing efficacy by a progressive ratio (PR) challenge. Following acquisition under FR conditions, rats received a PR heroin challenge, with RAP-103 (1 mg/kg, IP) or a vehicle injected 30 min before the challenge session. In the PR challenge experiments, RAP-103 reduced the reinforcing efficacy of heroin (e.g., lowered break point), which indicates that RAP-103 reduced the motivation to respond for heroin and suggests that RAP-103 has reduced the abuse liability of opioids. The Y-axis in FIG. 4B shows the break-point which was the number of lever presses the animal made until the reinforcer (heroin) was administered. Data are expressed as heroin PR breakpoint+S.E.M. * P<0.05 compared to VEH (vehicle) treatment.

FIGS. 5A-5B show RAP-103 did not affect sucrose intake in self-administration assays. FIG. 5A shows rats tested for 10 days with sucrose self-administration (SA) on a Fixed ratio (FR) schedule. Rats underwent sucrose acquisition (sucrose pellets) under a FR reinforcement schedule for at least 20 days. RAP-103 (1 mg/kg, IP) or vehicle (VEH) was injected 30 min before each sucrose SA session. Data are expressed as mean infusions per SA session (2 h)+S.E.M. N=6 rats/group. FIG. 5B shows reinforcing efficacy by a progressive ratio (PR) challenge. Following acquisition under FR conditions, rats received a PR sucrose challenge, with RAP-103 (1 mg/kg, IP) or VEH injected 30 min before the challenge session. The Y-axis shows the break-point which was the number of lever presses the animal made make until the reinforcer (sucrose) was administered. Data are expressed as sucrose PR breakpoint+S.E.M.

FIGS. 6A-6C show RAP-103 effects in chemokine receptor protein levels in the ventral tegmental area (VTA), nucleus accumbens (NAC) and prefrontal cortex (PFC) during morphine abstinence. Rats were treated for 10 days with morphine (5 mg/kg, SC) or saline. RAP-103 (1 mg/kg, IP) or a vehicle was administered 30 min before each morphine or saline injection. Brains were extracted 24 h following the last morphine (or saline) treatment for protein analysis (Western blot analysis) of chemokine receptors (CCR2, CXCR4 and CCR5) in the VTA, NAC and PFC. Results for the VTA region are shown in FIG. 6A. Results for the NAC region are shown in FIG. 6B. And results for the PFC region are shown in FIG. 6C. For each chemokine receptor, data are expressed as relative ratio (chemokine/marker/GADPH)+S.E.M. N=8 rats/group. ***P<0.001**P<0.01, or *P<0.05 compared to drug-naïve control rats (VEH+SAL group). ++P<0.01 or +P<0.05 compared to rats treated with morphine by itself (VEH+MOR group). Boxes: Representative protein bands from the different groups that includes the chemokine receptors (CCR2, CXCR4 and CCR5), OPRM1 (mu-opioid receptor) and TH (tyrosine hydroxylase) are also shown in FIGS. 6A-6C. RAP-103 normalized deficits in chemokine receptor protein levels in the NAC during morphine abstinence

FIGS. 7A-7C show RAP-103 effects on changes in mu-opioid (OPRM1) and tyrosine hydroxylase (TH) protein levels during morphine abstinence. Rats were treated for 10 days with morphine (5 mg/kg, SC) or saline. RAP-103 (1 mg/kg, IP) or a vehicle was administered 30 min before each morphine or saline injection. Brains were extracted 24 h following the last morphine (or saline) treatment for protein analysis (Western blot analysis) of mu-opioid (OPRM1) receptors and tyrosine hydroxylase (TH) in the VTA, NAC and PFC. Results for the VTA region are shown in FIG. 7A, results for NAC region are shown in FIG. 7B, and results for PFC region are shown in FIG. 7C. For each target, data are expressed as relative ratio (target/marker/GADPH)+S.E.M. N=8 rats/group. **P<0.01 compared to drug-naïve control rats (VEH+SAL group). ++P<0.01 or +P<0.05 compared to rats treated with morphine by itself (VEH+MOR group).

FIGS. 8A-8B show RAP-103 effects on IBA1 and GFAP levels in brane tissue. Rats were treated for 10 days with morphine (5 mg/kg, SC) or saline. RAP-103 (1 mg/kg, IP) or a vehicle was administered 30 min before each morphine or saline injection. Brains were extracted 24 h following the last morphine (or saline) treatment and gene expression studies were conducted to analyze astrocytic (IBA1) and microglial (IBA1) reactivities in the VTA, NAC and PFC. Results for GFAP mRNA (astrocytes) are shown in FIG. 8A. Results for IBA1 mRNA (microglia) are shown in FIG. 8B. For each target, data are expressed as fold-change+S.E.M. N=8 rats/group. *P<0.05 compared to drug-naïve control rats (VEH+SAL group). +P<0.05 compared to rats treated with morphine by itself (VEH+MOR group). RAP-103 normalizes enhancement of astrocyte reactivity in the NAC during morphine abstinence.

FIG. 9 shows an exemplary peptide manufacturing process for RAP-103. Figure discloses SEQ ID NO: 1.

FIGS. 10A-10B shows graphs of the pharmacokinetic properties of RAP-103 (R103). FIG. 10A shows the pharmacokinetic properties of RAP-103 with rapid brain entry by oral and intravenous dosing in rats and guinea pigs. FIG. 10B shows the plasma levels of RAP-103 for hours in non-human primates (monkeys).

### DETAILED DESCRIPTION

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various aspects may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the term ‘about’ a number can refer to that number plus or minus 10% of that number. The term ‘about’ a range can refer to that range minus 10% of its lowest value and plus 10% of its greatest value.

As used in the specification and claims, the singular forms “a”, “an” and “the” include plural references unless the context clearly dictates otherwise. For example, the term “a sample” includes a plurality of samples, including mixtures thereof.

The terms “determining”, “measuring”, “evaluating”, “assessing,” “assaying,” and “analyzing” are often used interchangeably herein to refer to forms of measurement and include determining if an element may be present or not (for example, detection). These terms may include quantitative, qualitative or quantitative, and qualitative determinations. Assessing may be alternatively relative or absolute. “Detecting the presence of” includes determining the amount of something present, as well as determining whether it may be present or absent.

The terms “subject,” “individual,” or “patient” are often used interchangeably herein. A “subject” may be a biological entity. The biological entity may be a plant, animal, or microorganism, including, for example, bacteria, viruses, fungi, and protozoa. The subject may be tissues, cells and their progeny of a biological entity obtained in vivo or cultured in vitro. The subject may be a mammal. The mammal may be a human. The subject may be diagnosed or suspected of being at high risk for a disease. In some cases, the subject may not be necessarily diagnosed or suspected of being at high risk for the disease.

The term “at least partially” may refer to a qualitative condition that exhibits a partial range or degree of a feature or characteristic of interest. For example, at least partially reducing opioid-induced respiratory depression in a subject may comprise a reduction in opioid-induced respiratory depression that is at least about: 5%, 10%, 20%, 30%, 40%, 50%, 60%, 70%, 75%, 80%, 85%, 90%, 95%, 99%, or 100% reduced relative to a subject being untreated.

The term “in vivo” may be used to describe an event that takes place in a subject's body.

The term “ex vivo” may be used to describe an event that takes place outside of a subject's body. An “ex vivo” assay may not be performed on a subject. Rather, it may be performed upon a sample separate from a subject. An example of an “ex vivo” assay performed on a sample may be an “in vitro” assay.

The term “in vitro” may be used to describe an event that takes place contained in a container for holding laboratory reagent such that it may be separated from the living biological source organism from which the material may be obtained. In vitro assays may encompass cell-based assays in which cells alive or dead are employed. In vitro assays may also encompass a cell-free assay in which no intact cells are employed.

As used herein, the terms “treatment” or “treating” are used in reference to a pharmaceutical or other intervention regimen for obtaining beneficial or desired results in the recipient such as preventing symptoms of an opioid-induced respiratory depression. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit may be achieved with the eradication or amelioration of one or more of the physiological symptoms associated with the underlying disorder such that an improvement may be observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For a prophylactic benefit, a subject at risk of developing a particular disease, or to a subject reporting one or more of the physiological symptoms of a disease may undergo treatment, even though a diagnosis of this disease may not have been made.

As used herein, a “dose” can refer to a measured quantity of a therapeutic agent to be taken at one time.

As used herein, the term “unit dose” or “dosage form” may be used interchangeably and may be meant to refer to pharmaceutical drug products in the form in which they are marketed for use, with a specific mixture of active ingredients and inactive components or excipients, in a particular configuration, and apportioned into a particular dose to be delivered. The term “unit dose” may also sometimes encompass non-reusable packaging. More than one unit dose may refer to distinct pharmaceutical drug products packaged together, or to a single pharmaceutical drug product containing multiple drugs and/or doses. The term “unit dose” may also sometimes refer to the particles comprising a pharmaceutical composition, and to any mixtures involved. Types of unit doses may vary with the route of administration for drug delivery, and the substance(s) being delivered. A solid unit dose may be the solid form of a dose of a chemical compound used as a pharmaceutically acceptable drug or medication intended for administration or consumption.

As used herein, “pharmaceutically acceptable salt” may refer to pharmaceutical drug molecules, which may be formed as a weak acid or base, chemically made into their salt forms, most frequently as the hydrochloride, sodium, or sulfate salts. Drug products synthesized as salts may enhance drug dissolution, boost absorption into the bloodstream, facilitate therapeutic effects, and increase its effectiveness. Pharmaceutically acceptable salts may also facilitate the development of controlled-release dosage forms, improve drug stability, extend shelf life, enhance targeted drug delivery, and improve drug effectiveness.

The phrase “pharmaceutically acceptable excipient” as used herein may refer to a pharmaceutically acceptable material, composition or vehicle, such as a liquid or solid filler, diluent, excipient, carrier, solvent or encapsulating material.

As used herein, “agent” or “biologically active agent” may refer to a biological, pharmaceutical, or chemical compound or a salt of any of these. Non-limiting examples may include a simple or complex organic or inorganic molecule, a peptide, a protein, a nucleotide such as an engineered RNA, an engineered DNA, an alternative nucleic acid, a protein, a carbohydrate, a toxin, or a chemotherapeutic compound. Various compounds may be synthesized, for example, small molecules and oligomers (e.g., oligopeptides and oligonucleotides), or synthetic organic compounds based on various core structures. In addition, various natural sources may provide compounds for screening, such as plant or animal extracts, and the like.

As used herein, the terms “effective amount” or “therapeutically effective amount” of a drug used to treat a disease may be an amount that may reduce the severity of a disease, reduce the severity of one or more symptoms associated with the disease or its treatment, or delay the onset of more serious symptoms or a more serious disease that may occur with some frequency following the treated condition. An “effective amount” may be determined empirically and in a routine manner, in relation to the stated purpose.

As used herein, “time to peak plasma concentration” can refer to the time required for a drug to reach peak concentration in plasma. Peak concentration in plasma can be defined as the plasma concentration that a drug achieves in a specified compartment or test area of the body after the drug has been administered and before the administration of a second dose.

The term “substantially” or “essentially” can refer to a qualitative condition that exhibits an entire or nearly total range or degree of a feature or characteristic of interest. In some cases, substantially can refer to a total range or degree of a feature or characteristic of interest by about plus or minus: 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. In some cases, substantially can refer to at least about: 70%, 75%, 80%, 85%, 90%, 95%, 99%, or 100% of the total range or degree of a feature or characteristic of interest.

As used herein, “HPLC” can refer to high-performance liquid chromatography (formerly referred to as high-pressure liquid chromatography), which is a technique in analytical chemistry used to separate, identify, and quantify each component in a mixture. HPLC can be a common technique used in pharmaceutical development, as it can be a method to ensure product purity.

The terms peptide and polypeptide can be used interchangeably herein.

The term “fragment,” as used herein, may be a portion of a sequence, a subset that may be shorter than a full-length sequence. A fragment may be a portion of a gene. A fragment may be a portion of a peptide or protein. A fragment may be a portion of an amino acid sequence. A fragment may be a portion of an oligonucleotide sequence. A fragment may be less than about: 20, 30, 40, 50 amino acids in length. A fragment may be about 10%, about 15%, about 20%, about 25%, about 30%, about 35%, about 40%, about 50%, about 60% or about 70% of the total length of an amino acid sequence or a nucleotide sequence. A fragment may be less than about: 20, 30, 40, 50 oligonucleotides in length.

The section headings used herein are for organizational purposes only and are not to be construed as limiting the subject matter described.

### Overview

Disclosed herein are compositions (e.g., pharmaceutical formulations), methods of treatment of a disease, and kits comprising a pharmaceutical formulation and a container. The compositions and formulations herein can be used to treat a subject with an opioid-induced respiratory depression.

Chemokine-opioid crosstalk has emerged as a physiological crossroads for therapeutic and adverse effects of opioids. Activation of chemokine receptors, for example, at least one of CCR2, CCR5 and CXCR4, can desensitize mu-opioid receptors and reduce opioid-induced antinociception. In turn, chemokine receptor antagonism can enhance opioid antinociception.

According to the National Institute on Drug Abuse, approximately 130 people succumb to an opioid overdose every day in the United States. The repercussions of substance use disorders echo far beyond the individual patients to also harshly impact families and social networks. Unfortunately, despite years of research, current treatments are limited and often ineffective, evidenced by the fact that relapse rates have remained discouragingly high. Thus, novel therapeutics for opioid use disorder (OUD) and treatment of opioid-induced respiratory depression is of high scientific priority.

Chemokine ligands can be small peptides that can contain from 70-125 amino acids and have molecular masses between 6-14 kD. As a subcategory of the greater cytokine signaling molecule family, chemokines can be distinct because they can use chemical gradients to carry out their signaling activities. Chemokine ligands have a wide variety of biological effects that can be engaged subsequent to their binding on an appropriate chemokine receptor. Chemokine receptors can be G-protein coupled 7 transmembrane (7TM) receptors that exist on a multitude of immune cell types. These ligands and receptors can work in concert to promote inflammation responses at sites of injury throughout the peripheral and central nervous systems.

Chemokines have been studied as inflammatory signaling molecules, although a smaller subset have been more recently implicated as master regulators of various homeostatic mechanism. The inflammatory class of chemokines can be upregulated by inflammatory factors such as proinflammatory cytokines, microbes, and trauma. Once activated, they can work to recruit circulating leukocytes to a site of injury by binding to the chemokine receptors on the leukocytes. In a process known as chemotaxis, the leukocytes can then follow a gradient of inflammatory chemokines to the site of inflammation and orchestrate an appropriate immune response on the inflamed tissue. The expression of inflammatory chemokines can be induced temporarily and can return to baseline levels upon resolution of the inflammation.

Chemokine receptor antagonism has high potential as a pharmacotherapy because, for example, chemokines can act on 7-transmembrane (7TM) receptors. Blocking 7TM receptors can work because the binding sites can be narrow enough to block with small molecules. Exciting preclinical work has already shown that multiple sclerosis phenotypes can be avoided by knocking out CCR1 and CCR2, implicating these receptors as potential treatment targets for MS.

Presently, two existing chemokine receptor antagonists (CRAs) have been approved for clinical uses. The first is maraviroc, which inhibits the entry of the human immunodeficiency virus into cells that express CCR5. Secondly, AMD3100 is an antagonist of CXCR4, and works to mobilize hematopoietic stem cells in order to complete successful organogenesis of the bone marrow.

The chemokine system and the opioid system can engage in ample crosstalk, but the impacts of this are complex. Chemokines can have multimodal influences on nociception, as they can act as communication regulators between immune, neuronal, and glial cell types, and also because they can be expressed all along nociceptive pathways.

Opioid receptors and chemokine receptors can co-express in the same microenvironment, or even on the same neuron, and they can influence each other's functions. Contradictory evidence shows that opioids can both encourage and discourage chemokine release and receptor viability. For example, opioids can exert their own proinflammatory functions by inducing chemotaxis of immune cells and also by stimulating chemokine receptors. In contrast to this, micro-opioid receptor agonists can downregulate chemokine induced chemotaxis. In reverse, chemokines have been shown to comprise the endogenous opioid system. Proinflammatory chemokines (CCL2, CCL3, CCL5, and CXCL8) can both desensitize and induce internalization of the mu opioid receptor; and tolerance to opioids can be caused by changes in opioid receptor expression.

Further research in support of chemokine regulation of opioid receptors has shown that minocycline, which inhibits chemokine release, attenuates tolerance to morphine. Another study found that endogenous opioid release at injured tissues can be enhanced by the activation of CXCR2 by CXCL2 or CXCL3. Additionally, spinal injections of CXCL1 or CXCL12, which were found to be upregulated in opioid-tolerant human patients, accelerated morphine tolerance when administered before a rodent tail-flick assay. Lastly, while CRAs on their own do not seem to possess any pain-relieving properties, co-administration of existing CRA's with opioids has been shown to enhance the analgesic effects of opioids by shifting the dose response curve to the left.

Other related research demonstrated that a bivalent ligand that was artificially synthesized to act as an agonist for the mu opioid receptor and an antagonist for CCR5 produced profound antinociception that was 2000-fold stronger than that of morphine alone. Thus, dynamic interplay between chemokine and opioid systems makes CRA's intriguing candidates for the treatment of OUD or opioid use disorder.

RAP-103, works as a CRA and at least partially inhibits chemokine ligands from binding to four specific chemokine receptors: CCR2, CCR5, CCR8, and CXCR4. RAP-103 exhibits significant oral bioavailability (>88% in primates), with rapid CNS entry, and may, for example, require 1 dose per day. Previous research demonstrates that RAP-103 can attenuate behavioral hallmarks of neuropathic pain, such as mechanical allodynia and thermal hyperalgesia. In support of this, RAP-103 can also downregulate corresponding molecular hallmarks of neuropathic pain such as phagocytosis, immune cell migration, and cell membrane ruffling.

While RAP-103 and other CRAs have been studied in the context of pain, there is little known about their potential uses for treating opioid-induced respiratory depression. Opioid-induced respiratory depression is particularly important because it is the primary cause of death due to opioid use for pain control as well as for abuse. Drug overdose remains the leading cause of accidental death in the United States, with opioids being the most common drug, and the CDC currently estimates more than 1000 emergency department visits daily related to the misuse of opioids and about 91 opioid overdose deaths every day.

In addition to respiratory depression, dependence results from chronic opioid use. Approximately 16 million individuals worldwide have had or currently suffer from opioid use disorder (OUD), and more than 500,000 in the United States alone are dependent on heroin. Approved treatments (buprenorphine and methadone) for OUD are opioids that carry a risk of abuse liability. Thus, it is important to investigate non-opioid based approaches, such as chemokine receptor antagonism studied here, to identify effective and safe non-opioid based treatments.

In some instances, a peptide herein may comprise up to eight or more than eight contiguous amino acids or derivatives thereof, comprising the general formula A-B-C-E-F-G-H-I, and wherein: A is D-Ala, or a derivative thereof; B is D-Ser, or D-Thr, or a derivative of any of these; C is D-Ser, or D-Thr, or a derivative of any of these; E is D-Ser, D-Thr, D-Asn, D-Glu, D-Arg, D-Ile, or D-Leu, or a derivative of any of these; F is D-Ser, D-Thr, D-Asp, or D-Asn, or a derivative of any of these; G is D-Thr, D-Ser, D-Asn, D-Arg, D-Gln, D-Lys, or D-Trp, or a derivative of any of these; His D-Tyr, or a derivative thereof, and I is D-Thr, D-Ser, D-Arg, or Gly or a derivative of any of these. In some cases, a peptide herein can comprise at least five contiguous amino acids or derivatives thereof comprising the general formula: E-F-G-H-I, wherein: E is D-Ser, D-Thr, D-Asn, D-Glu, D-Arg, D-Ile, or D-Leu, or a derivative of any of these; wherein F is D-Ser, D-Thr, D-Asp, or D-Asn, or a derivative of any of these; wherein G is D-Thr, D-Ser, D-Asn, D-Arg, D-Gln, D-Lys, or D-Trp, or a derivative of any of these; wherein H is D-Tyr, or a derivative thereof; and wherein I is D-Thr, D-Ser, D-Arg, or Gly, or a derivative of any of these.

In some embodiments, peptides in a composition may be used to treat opioid-induced respiratory depression.

The multi-chemokine receptor antagonist peptide RAP-103 (All D-peptide-Thr-Thr-Asn-Tyr-Thr (SEQ ID NO: 1)) is being developed to provide a therapy to reverse opioid-induced respiratory depression. Surprisingly, RAP-103 was able to treat opioid-induced respiratory depression in an animal model by increasing blood oxygen levels and decreasing the respiratory rate of animals that had been administered an opioid or a salt thereof.

### Peptides

As used herein, amino acids can be referenced by their one or three letter codes, which are shown in Table 1.

TABLE 1
Amino acid

| | | |
|---|---|---|
| Alanine | Ala | A |
| Arginine | Arg | R |
| Asparagine | Asn | N |
| Aspartic Acid | Asp | D |
| Cysteine | Cys | C |
| Glutamic Acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Amino acids, depending upon the configuration at the alpha carbon, can be D or L-excepting glycine which does not contain four non-identical substituents on its alpha carbon atom. The designations D and L should not be confused with one letter amino acid codes. In some embodiments, D amino acids are designated with a D in front of the amino acid (e.g., D-Ser, dA). If the amino acid has four non-identical substituents on its alpha carbon atom, and the amino acid is not designated with a D in front of the amino acid, the amino acid can be of the L configuration. The amino acid glycine, lacking four non-identical substituents on its alpha carbon atom, may not be D or L.

Peptides herein can include peptides in Table 2.

TABLE 2
Peptide sequences
Peptide Sequences

TTNYT (SEQ ID NO: 1)
(referred to herein
as RAP-103)
SSTYR (SEQ ID NO: 2)
STNYT (SEQ ID NO: 3)
NTSYG (SEQ ID NO: 4)
dASTTTNYT-NH2 (SEQ ID NO: 5)
(referred to herein as DAPTA)
dASTTTNYT (SEQ ID NO: 6)
ASTTTNYT-amide (SEQ ID NO: 7)
ASTTTNYT (SEQ ID NO: 8)
(referred to herein
as RAP-310)
ETWYS (SEQ ID NO: 9)

Wherein in Table 2, peptides TTNYT (SEQ ID NO: 1), SSTYR (SEQ ID NO: 2), STNYT (SEQ ID NO: 3) and ASTTTNYT (SEQ ID NO: 8) are all D-peptides such that each amino acid in the peptide is in the D configuration. Wherein in the table, each amino acid of peptide NTSYG, except for glycine, is in the D configuration (SEQ ID NO: 4). Wherein in the table, each amino acid of peptide ETWYS is in the D configuration (SEQ ID NO: 9). Wherein in the table, dASTTTNYT the alanine is in the D configuration, and all other amino acids in the peptide are in the L configuration (SEQ ID NO: 6). And wherein in the table, dASTTTNYT-NH2 the alanine is in the D configuration, and all other amino acids in the peptide are in the L configuration (SEQ ID NO: 5). In some cases, a peptide such as TTNYT (SEQ ID NO: 1) can comprise a N-terminal methyl group (e.g., N-methyl-TTNYT (SEQ ID NO: 10)). In some cases, a peptide can comprise an amide. For example ASTTTNYT (SEQ ID NO: 8) can be ASTTTNYT-amide (SEQ ID NO: 7). In some embodiments, DAPTA comprises dASTTTNYT-NH2 (SEQ ID NO: 5).

In some cases, peptides TTNYT (SEQ ID NO: 11), SSTYR (SEQ ID NO: 12), STNYT (SEQ ID NO: 13) and ASTTTNYT (SEQ ID NO: 14) can be all L-peptides such that each amino acid in the peptide is in the L configuration

### Peptide Compositions

In some instances, a composition is provided which comprises a peptide wherein the peptide comprises at least five contiguous amino acids or derivatives thereof comprising the general formula: E-F-G-H-I, wherein: E is D-Ser, D-Thr, D-Asn, D-Glu, D-Arg, D-Ile, or D-Leu, or a derivative of any of these; wherein F is D-Ser, D-Thr, D-Asp, or D-Asn, or a derivative of any of these; wherein G is D-Thr, D-Ser, D-Asn, D-Arg, D-Gln, D-Lys, or D-Trp, or a derivative of any of these; wherein H is D-Tyr, or a derivative thereof; and wherein I is D-Thr, D-Ser, D-Arg, or Gly, or a derivative of any of these.

In some instances, E can be D-Thr. In some instances, F can be D-Thr. In some instances, E can be D-Thr and F can be D-Thr. In some instances, G can be D-Asn. In some instances, F can be D-Thr and G can be D-Asn. In some instances, E can be D-Thr, F can be D-Thr, and G can be D-Asn. In some instances, H can be D-Tyr. In some instances, E can be D-Thr and H can be D-Tyr. In some instances, G can be D-Asn and H can be D-Tyr. In some instances, I can be D-Thr. In some instances, E can be D-Thr, F can be D-Thr, G can be D-Asn, H can be D-Tyr, and I can be D-Thr.

In some instances, the peptide may comprise at least eight contiguous amino acids or derivatives thereof, comprising the general formula A-B-C-E-F-G-H-I, and wherein: A is D-Ala, or a derivative thereof; B is D-Ser, or D-Thr, or a derivative of any of these; C is D-Ser, or D-Thr, or a derivative of any of these; E is D-Ser, D-Thr, D-Asn, D-Glu, D-Arg, D-Ile, or D-Leu, or a derivative of any of these; F is D-Ser, D-Thr, D-Asp, or D-Asn, or a derivative of any of these; G is D-Thr, D-Ser, D-Asn, D-Arg, D-Gln, D-Lys, or D-Trp, or a derivative of any of these; H is D-Tyr, or a derivative thereof, and I is D-Thr, D-Ser, D-Arg, or a derivative of any of these.

In some cases, A can be D-Ala. In some instances, B can be D-Ser. In some instances, B can be D-Thr. In some instances, A can be D-Ala and B can be D-Ser. In some instances, A can be D-Ala and B can be D-Thr. In some instances, C can be D-Ser. In some instances, C can be D-Thr. In some instances, B can be D-Ser and C can be D-Ser. In some instances, B can be D-Thr and C can be D-Ser. In some instances, B can be D-Thr and C can be D-Thr. In some instances, E can be D-Thr. In some instances, F can be D-Thr. In some instances, E can be D-Thr and F can be D-Thr. In some instances, G can be D-Asn. In some instances, F can be D-Thr and G can be D-Asn. In some instances, E can be D-Thr, F can be D-Thr, and G can be D-Asn. In some instances, H can be D-Tyr. In some instances, E can be D-Thr and H can be D-Tyr. In some instances, G can be D-Asn and H can be D-Tyr. In some instances, I can be D-Thr. In some instances, E can be D-Thr, F can be D-Thr, G can be D-Asn, H can be D-Tyr, and I can be D-Thr.

In some embodiments, a peptide at the C terminus can be esterified, glycosylated, or amidated at the C terminus. In some cases, I or a derivative thereof can be esterified, glycosylated, or amidated at the C terminus.

In some instances, the peptide sequence can comprise a sequence of ASTTTNYT, where each amino acid, individually, can be of the L or of the D configuration (SEQ ID NO: 15); in some instances, all amino acids in the sequence ASTTTNYT can be in the L configuration (SEQ ID NO: 14); in some instances, all amino acids in the sequence ASTTTNYT can be of the D configuration (SEQ ID NO: 8).

In some instances, the peptide sequence can comprise a sequence of SSTYR, where each amino acid, individually, can be of the L or of the D configuration (SEQ ID NO: 16); in some instances, all amino acids in the sequence SSTYR can be in the L configuration (SEQ ID NO: 12); in some instances, all amino acids in the sequence SSTYR can be of the D configuration (SEQ ID NO: 2).

In some instances, the peptide sequence can comprise a sequence of STNYT, where each amino acid, individually, can be of the L or of the D configuration (SEQ ID NO: 17); in some instances, all amino acids in the sequence STNYT can be in the L configuration (SEQ ID NO: 13); in some instances, all amino acids in the sequence STNYT can be of the D configuration (SEQ ID NO: 3).

In some instances, the peptide sequence can comprise a sequence of NTSYG, where each amino acid, individually, can be of the L or of the D configuration (SEQ ID NO: 18); in some instances, all amino acids in the sequence NTSYG can be in the L configuration (SEQ ID NO: 19); in some instances, all amino acids in the sequence NTSYG can be of the D configuration (SEQ ID NO: 4).

In some instances, the peptide sequence can comprise a sequence of TTNYT, where each amino acid, individually, can be of the L or of the D configuration (SEQ ID NO: 20); in some instances, all amino acids in the sequence TTNYT can be in the L configuration (SEQ ID NO: 11); in some instances, all amino acids in the sequence TTNYT can be of the D configuration (SEQ ID NO: 1). ASTTTNYT-NH2, where each amino acid, individually, can be of the L or of the D configuration (SEQ ID NO: 21); in some instances, all amino acids in the sequence ASTTTNYT-NH2 can be in the L configuration (SEQ ID NO: 22); in some instances, all amino acids int the sequence ASTTTNYT-NH2 can be of the D configuration (SEQ ID NO: 23). In some instances, the A in the sequence ASTTTNYT-NH2 can be in the D configuration and all other amino acids in this sequence can be in the L configuration (SEQ ID NO: 5). In the sequence, the —NH₂ designations that the amino acid threonine in the C terminal end of the sequence has an amide as opposed to a carboxylic acid.

In some instances, the peptide sequence is a multi-chemokine receptor antagonist RAP-103 (R103) (All D-peptide-Thr-Thr-Asn-Tyr-Thr (SEQ ID NO: 1)). In some instances, the peptide sequence is a multi-chemokine receptor antagonist DAPTA (dASTTTNYT-NH₂ (SEQ ID NO: 5)).

In some instances, the peptide described herein can be in the form of a pharmaceutically acceptable salt, such as acetate.

### Pharmaceutical Compositions

An active pharmaceutical ingredient may be any substance or mixture of substances intended to be used in the manufacture of a drug (medicinal) product and that, when used in the production of a drug, becomes an active ingredient of the drug product. Such substances may be intended to furnish pharmacological activity or other direct effect in the diagnosis, cure, mitigation, treatment, or prevention of disease or to affect the structure or function of the body. In some embodiments, compositions herein comprise peptides as an active pharmaceutical ingredient. In some cases, compositions herein comprise pharmaceutical compositions.

In some embodiments, a peptide described herein when stored in a sealed container placed in a room at about: 20 °C, 21 °C, 22 °C, 23 °C, 24 °C, 25 °C, 26 °C, 27 °C, 28 °C, 29 °C, or 30 °C, and a room atmosphere having about: 40 percent, 45 percent, 50 percent, 55 percent, or 60 percent relative humidity, retains at least about: 80%, 90%, 95%, 96%, 97%, 98%, 99%, or 100% of the active ingredient or the salt thereof after about: 1 month, 2 months, 3 months, 4 months, 5 months, 6 months, 7 months, 8 months, 9 months, 10 months, 11 months, or 12 months as measured by HPLC. In some cases, the peptide can be formulated as a pill. In some embodiments, a peptide described herein formulated as a liquid when stored in a sealed container placed in a room at about: 20 °C, 21 °C, 22 °C, 23 °C, 24 °C, 25 °C, 26 °C, 27 °C, 28 °C, 29 °C, or 30 °C, and a room atmosphere having about: 40 percent, 45 percent, 50 percent, 55 percent, or 60 percent relative humidity, retains at least about: 80%, 90%, 95%, 96%, 97%, 98%, 99%, or 100% of the active ingredient or the salt thereof after about: 1 month, 2 months, 3 months, 4 months, 5 months, 6 months, 7 months, 8 months, 9 months, 10 months, 11 months, or 12 months as measured by HPLC.

### Representative Acids for Salts

In some embodiments, the pharmaceutically acceptable salt of the peptide can be formed from the peptide and an acid. In some embodiments, the acid can be at least one of: 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), benzenesulfonic acid, benzoic acid, camphoric acid (+), camphor-10-sulfonic acid (+), capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, hydrobromic acid, hydrochloric acid, isobutyric acid, lactic acid (DL), lactobionic acid, lauric acid, maleic acid, malic acid (−L), malonic acid, mandelic acid (DL), methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid (−L), salicylic acid, sebacic acid, stearic acid, succinic acid, sulfuric acid, tartaric acid (+L), thiocyanic acid, toluenesulfonic acid (p), undecylenic acid, or any combination thereof.

### Representative Salts

In some embodiments, a salt herein can comprise a pharmaceutically acceptable salt. In some embodiments, the pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, phosphate, sulphate and the like; organic acid salts such as citrate, lactate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate and the like. In some instances, a salt of a peptide or derivative thereof or a compound can be a Zwitterionic salt.

In some aspects, the pharmaceutical composition comprising the salt of the pharmaceutically active ingredient, wherein the salt comprises an organic salt, an inorganic salt, or any combination thereof. In some cases, an organic salt may comprise a phosphinate (e.g., sodium hypophosphite), a hydrazinium salt, a urate, a diazonium salt, an oxalate salt, a tartrate, a choline chloride. An example of an inorganic salt may be sodium chloride, calcium chloride, magnesium chloride, sodium bicarbonate, potassium chloride, sodium sulfate, calcium carbonate, calcium phosphate, or any combination thereof.

In some aspects, the pharmaceutical composition comprising the salt of the pharmaceutically active ingredient, wherein the salt comprises an HCl salt, an ascorbic acid salt, a mandelic acid salt, an aspartic acid salt, a carbonic acid salt, a citric acid salt, a formic acid salt, a glutamic acid salt, a lactic acid salt, a lauric acid salt, a maleic acid salt, a borate salt, a bitartrate salt, a palmitic acid salt, a phosphoric acid salt, or any combination thereof.

In some aspects, the pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, phosphate, sulphate and the like; organic acid salts such as citrate, lactate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate and the like.

### Representative Excipients

In some embodiments, a carrier, a diluent, and/or an excipient, can include pharmaceutically acceptable carriers, diluents, and/or excipients. As used herein, “excipient” can refer to a substance formulated alongside the active ingredient of a medication, included for the purpose of long-term stabilization, bulking up solid formulations that contain potent active ingredients in small amounts, and/or to confer a therapeutic enhancement on the active ingredient(s) in the final dosage form. Excipients may facilitate drug absorption, reduce viscosity, or enhance solubility. Excipients may also facilitate the handling of the active ingredients, improve in vitro stability, and/or extend pharmaceutical product shelf life. Excipient selection may vary with the route of administration for drug delivery, the unit dose, as well as the active ingredients comprising the composition.

In some embodiments, a pharmaceutically acceptable excipient can comprise anhydrous calcium phosphate, dihydrate calcium phosphate, hydroxypropyl methylcellulose, croscarmellose sodium, GMO-free croscarmellose sodium, carbomers, magnesium aluminometasilicate, mannitol, povidone (PVP), crospovidone, sorbitol, dimethicone, sodium stearyl fumarate, sodium starch glycollate, hydroxypropylcellulose, native corn starch, modified corn starch, carrageenan, alginates, silicon dioxide, microcrystalline cellulose, carboxymethylcellulose sodium, alginates, carboxymethylcellulose (CMC), sodium carboxymethylcellulose (Na CMC), carbomers, natural gums, sorbitol, maltitol, glucose syrup, silicones, carbomers, fatty alcohols, alcohols, carbohydrates, petrolatum derivatives, butters, waxes, DMSO Procipient®, esters, fatty acids, oil-in-water (O/W) emulsifiers, water-in-oil (W/O) emulsifiers, silicas, fumed silicas, polysorbates, isopropyl myristate, cellulosic derivates, xanthan gum, propylenglycol, noveon AA-1 polycarbophyl, dimethyl isosorbate, polysilicone elastomer 1100, polysilicone elastomer 1148P, preservatives, flavors, colors, functional coatings, aesthetic coatings, a pharmaceutically acceptable salt of any of these, or any combination thereof.

In some embodiments, a pharmaceutically acceptable excipient can comprise acacia, acesulfame potassium, acetic acid, glacial, acetone, acetyl tributyl citrate, acetyl triethyl citrate, agar, albumin, alcohol, alginic acid, aliphatic polyesters, alitame, almond oil, alpha tocopherol, aluminum hydroxide adjuvant, aluminum oxide, aluminum phosphate adjuvant, aluminum stearate, ammonia solution, ammonium alginate, ascorbic acid, ascorbyl palmitate, aspartame, attapulgite, bentonite, benzalkonium chloride, benzethonium chloride, benzoic acid, benzyl alcohol, benzyl benzoate, boric acid, bronopol, butylated hydroxyanisole, butylated hydroxytoluene, butylparaben, calcium alginate, calcium carbonate, calcium phosphate, dibasic anhydrous, calcium phosphate, dibasic dihydrate, calcium phosphate, tribasic, calcium stearate, calcium sulfate, canola oil, carbomer, carbon dioxide, carboxymethylcellulose calcium, carboxymethylcellulose sodium, carrageenan, castor oil, castor oil, hydrogenated, cellulose (e.g. microcrystalline, powdered, silicified microcrystalline, acetate, acetate phthalate) ceratonia, cetostearyl alcohol, cetrimide, cetyl alcohol, cetylpyridinium chloride, chitosan, chlorhexidine, chlorobutanol, chlorocresol, chlorodifluoroethane, chlorofluorocarbons, chloroxylenol, cholesterol, citric acid monohydrate, colloidal silicon dioxide, coloring agents, copovidone, corn oil, cottonseed oil, cresol, croscarmellose sodium, crospovidone, cyclodextrins, cyclomethicone, denatonium benzoate, dextrates, dextrin, dextrose, dibutyl phthalate, dibutyl sebacate, diethanolamine, diethyl phthalate, difluoroethane, dimethicone, dimethyl ether, dimethyl phthalate, dimethyl sulfoxide, dimethylacetamide, disodium edetate, docusate sodium, edetic acid, erythorbic acid, erythritol, ethyl acetate, ethyl lactate, ethyl maltol, ethyl oleate, ethyl vanillin, ethylcellulose, ethylene glycol palmitostearate, ethylene vinyl acetate, ethylparaben, fructose, fumaric acid, gelatin, glucose, glycerin, glyceryl behenate, glyceryl monooleate, glyceryl monostearate, glyceryl palmitostearate, glycofurol, guar gum, hectorite, heptafluoropropane, hexetidine, hydrocarbons, hydrochloric acid, hydroxyethyl cellulose, hydroxyethylmethyl cellulose, hydroxypropyl cellulose, hydroxypropyl cellulose, low-substituted, hydroxypropyl starch, hypromellose, hypromellose acetate succinate, hypromellose phthalate, honey, imidurea, inulin, iron oxides, isomalt, isopropyl alcohol, isopropyl myristate, isopropyl palmitate, kaolin, lactic acid, lactitol, lactose, anhydrous, lactose, monohydrate, lactose, spray-dried, lanolin, lanolin alcohols, lanolin, hydrous, lauric acid, lecithin, leucine, linoleic acid, macrogol hydroxystearate, magnesium aluminum silicate, magnesium carbonate, magnesium oxide, magnesium silicate, magnesium stearate, magnesium trisilicate, malic acid, maltitol, maltitol solution, maltodextrin, maltol, maltose, mannitol, medium-chain triglycerides, meglumine, menthol, methylcellulose, methylparaben, mineral oil, mineral oil, light, mineral oil and lanolin alcohols, monoethanolamine, monosodium glutamate, monothioglycerol, myristic acid, neohesperidin dihydrochalcone, nitrogen, nitrous oxide, octyldodecanol, oleic acid, oleyl alcohol, olive oil, palmitic acid, paraffin, peanut oil, pectin, petrolatum, petrolatum and lanolin alcohols, phenol, phenoxyethanol, phenylethyl alcohol, phenylmercuric acetate, phenylmercuric borate, phenylmercuric nitrate, phosphoric acid, polacrilin potassium, poloxamer, polycarbophil, polydextrose, polyethylene glycol, polyethylene oxide, polymethacrylates, poly(methyl vinyl ether/maleic anhydride), polyoxyethylene alkyl ethers, polyoxyethylene castor oil derivatives, polyoxyethylene sorbitan fatty acid esters, polyoxyethylene stearates, polyvinyl acetate phthalate, polyvinyl alcohol, potassium alginate, potassium benzoate, potassium bicarbonate, potassium chloride, potassium citrate, potassium hydroxide, potassium metabisulfite, potassium sorbate, povidone, propionic acid, propyl gallate, propylene carbonate, propylene glycol, propylene glycol alginate, propylparaben, 2-pyrrolidone, raffinose, saccharin, saccharin sodium, saponite, sesame oil, shellac, simethicone, sodium acetate, sodium alginate, sodium ascorbate, sodium benzoate, sodium bicarbonate, sodium borate, sodium chloride, sodium citrate dihydrate, sodium cyclamate, sodium hyaluronate, sodium hydroxide, sodium lactate, sodium lauryl sulfate, sodium metabisulfite, sodium phosphate, dibasic, sodium phosphate, monobasic, sodium propionate, sodium starch glycolate, sodium stearyl fumarate, sodium sulfite, sorbic acid, sorbitan esters (sorbitan fatty acid esters), sorbitol, soybean oil, starch, starch (e.g. pregelatinized, sterilizable maize), stearic acid, stearyl alcohol, sucralose, sucrose, sugar, compressible, sugar, confectioner's sugar spheres, sulfobutylether b-cyclodextrin, sulfuric acid, sunflower oil, suppository bases, hard fat, talc, tartaric acid, tetrafluoroethane, thaumatin, thimerosal, thymol, titanium dioxide, tragacanth, trehalose, triacetin, tributyl citrate, triethanolamine, triethyl citrate, vanillin, vegetable oil, hydrogenated, water, wax, anionic emulsifying, wax (e.g. carnauba, cetyl esters, microcrystalline, nonionic emulsifying, white, yellow), xanthan gum, xylitol, zein, zinc acetate, zinc stearate, or any combination thereof.

In some embodiments, a pharmaceutically acceptable excipient can comprise a carbohydrate, an alginate, povidone, a carbomer, a flavor, a natural gum, a silicone, an alcohol, a butter, a wax, a fatty acid, a preservative, a pharmaceutically acceptable salt of any of these, or any combination thereof. In some embodiments, a pharmaceutically acceptable excipient can comprise a carbohydrate. In some embodiments, the carbohydrate can comprise lactose, microcrystalline cellulose, cellulose, mannitol, sorbitol, starch, starch glycolate, hydroxypropyl methylcellulose, hydroxypropyl methylcellulose acetate succinate, a cyclodextrin, maltodextrin, croscarmellose sodium, corn starch, carrageenan, sorbitol, maltitol, glucose, a pharmaceutically acceptable salt of any of these, or any combination thereof.

In some aspects, the weigh to weight ratio of: a) the pharmaceutically acceptable excipient and b) the active ingredient ranges from about 1:1 to about 10000:1. In some aspects, the weight to weight ratio of: a) the pharmaceutically acceptable excipient and b) the active ingredient ranges from about 1:1 to about 20:1, about 1:1 to about 15:1, about 1:1 to about 10:1, about 1:1 to about 5:1, about 1:1 to about 2:1, about 2:1 to about 20:1, about 2:1 to about 15:1, about 2:1 to about 10:1, about 2:1 to about 5:1, about 5:1 to about 20:1, about 5:1 to about 15:1, about 5:1 to about 10:1, about 10:1 to about 15:1, about 10:1 to about 20:1, about 15:1 to about 20:1, about 18:1 to about 25:1, or about 25:1 to about 30:1. In some aspects, the weight to weight ratio of: a) the pharmaceutically acceptable excipient and b) the active ingredient may be about: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, or 30:1 In some aspects, the weight to weight ratio of: a) the pharmaceutically acceptable excipient and b) the active ingredient ranges from about 1:1 to about 1:10, about 1:1 to about 1:8, about 1:1 to about 1:5, about 1:1 to about 1:2, about 1:2 to about 1:10, about 1:2 to about 1:8, about 1:2 to about 1:5, about 1:5 to about 1:10, about 1:5 to about 1:8, about 1:8 to about 1:10.

### Carriers

In some instances, a pharmaceutically acceptable carrier or diluent can comprise water. In some embodiments, the water can be sterile. In some embodiments, the water can contain a buffer, a carbohydrate, a salt, a pH adjuster, or any combination of these. Simple sugars such as mannitol, sucrose, glucose, or trehalose may be added to inhibit peptide or peptide aggregation, in amounts from 1 to 50 mgs/ml. Citrate can be used as a buffer. In some instances, sodium chloride and phosphate salts may or may not be employed. Larger polysaccharides may also be used to enhance stability. In some cases, a carrier can be a salt solution such as a saline.

In certain instances, a carrier may refer to reagents, cells, compounds, materials, compositions, dosage forms, or any combination thereof that can be compatible with agents that can be administered therapeutically. In some cases, a carrier can be suitable for use in contact with a tissue of a subject. In some cases, a carrier may not have a toxicity, an irritation, an allergic response, or any combination thereof. A carrier that may be suitable for use can include a liquid, a solid material (e.g., a pill, or a suppository) or any combination thereof. In some cases, a carrier can be designed to resist degradation within the body (non-biodegradable) or they may be designed to degrade within the body (biodegradable). A biodegradable material can further be bioresorbable or bioabsorbable. In some cases, a biodegradable material can be degraded and eliminated from the body by conversion into other materials or breakdown and elimination through natural pathways.

### Oral Bioavailability

In some instances, the peptides, derivatives thereof, or salts of any of these can be orally bioavailable. In some cases, the pharmaceutical composition is formulated for oral administration. The percent oral bioavailability can be at least, at least about, or about: 5%, 10% 20%, 30%, 40%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% after administration. In some cases, a pharmaceutical composition herein can be formulated as a pill, a capsule, a liquid, or a tablet, for oral administration.

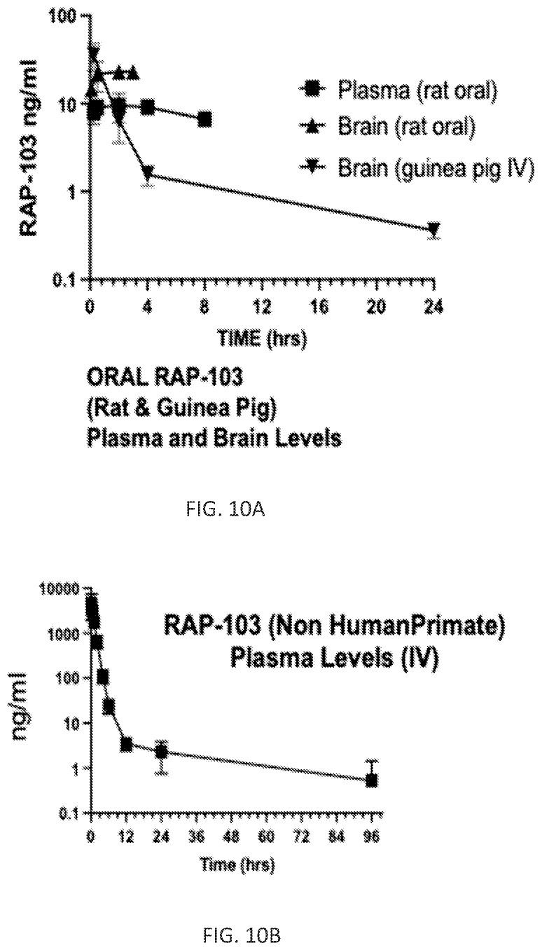

For example, RAP-103 quickly entered the brain by oral, and IV dosing in rodents and non-human primates (Rhesus macaque). The non-human primates showed oral bioavailability of 88%. RAP-103 preferentially entered the brain by oral compared to IV dosing (as shown in FIG. 10A).

In some instances, the peptides, derivatives, or salts of any of these, can be administered to a subject, who can be a subject in need thereof. In some embodiments, the subject has opioid-induced respiratory depression. In some embodiments, the subject can be a human, can be a male, or can be a female. In some instances, the subject can be under 18 years of age. In some instances, the subject can be over 18 years of age. In some cases, the subject can be a child, for example a subject from about: 0-18 years of age. In some cases, the subject can be an adult, for example a subject who is equal to, or greater than 18 years of age. In some instances, the subject can range from about 1 year of age to about 120 years of age. In some cases, a subject can be a subject who is equal to, or greater than 65 years of age. In some embodiments, a subject can be from about 1 day to about 10 months old, from about 9 months to about 24 months old, from about 1 year to about 8 years old, from about 5 years to about 25 years old, from about 20 years to about 50 years old, from about 40 years to about 80 years old, or from about 50 years to about 130 years old.

### Administration

In some embodiments, the terms “administer,” “administering”, “administration,” and the like, as used herein, can refer to methods that can be used to enable delivery of compounds, peptides, derivatives thereof, or salts of any of these, or compositions described herein, to the desired site of biological action. In some cases, delivery can include injection, inhalation, catheterization, gastrostomy tube administration, intravenous administration, intraosseous administration, ocular administration, otic administration, topical administration, transdermal administration, local administration, oral administration, rectal administration, nasal administration, intravaginal administration, intracavernous administration, transurethral administration, buccal administration, sublingual administration, or a combination thereof. In some cases, delivery can comprise oral administration, intranasal administration, or intravenous administration. In some cases, the pharmaceutical composition can be administered by: an oral route, an injection route, a sublingual route, a buccal route, a rectal route, a vaginal route, an ocular route, an otic route, a nasal route, an internasal route, an inhalation route, a cutaneous route, a subcutaneous route, an intramuscular route, an intravenous route, a systemic route, a local route, a transdermal route, or any combination thereof. In some cases, delivery can comprise delivery by an inhalation route for example by an inhaler. Delivery can include direct application to the affect tissue or region of the body. Delivery can include a parenchymal injection, an intra-thecal injection, an intra-ventricular injection, or an intra-cisternal injection. A composition provided herein can be administered by any method. A method of administration can be by intraarterial injection, intracerebroventricular injection, intracisternal injection, intramuscular injection, intraorbital injection, intraparenchymal injection, intraperitoneal injection, intraspinal injection, intrathecal injection, intravenous injection, intraventricular injection, stereotactic injection, subcutaneous injection, epidural, or any combination thereof. Delivery can include parenteral administration (including intravenous, subcutaneous, intrathecal, intraperitoneal, intramuscular, intravascular or infusion administration). In some embodiments, delivery can comprise a nanoparticle, a viral vector, a viral-like particle, a liposome, an exosome, an extracellular vesicle, a microrobot, a microneedle, an implant, or a combination thereof. In some cases, delivery can be from a device. In some instances, delivery can be administered by a pump, an infusion pump or a combination thereof. In some cases, delivery can be by an enema, an eye drop, a nasal spray, an ear drop, or any combination thereof. In some cases, delivery can comprise an inhaler, a diffuser, a nebulizer, or a combination thereof. Delivery can include topical administration (such as a lotion, a cream, a patch, a gel, a spray, a drip, a liquid formulation, an ointment) to an external surface of a surface, such as a skin. In some instances, a subject can administer the composition in the absence of supervision. In some instances, a subject can administer the composition under the supervision of a medical professional (e.g., a physician, nurse, physician's assistant, orderly, hospice worker, a first responder, etc.). In some cases, a medical professional can administer the composition. In some cases, the subject can administer the composition.

In some embodiments, administering can be performed at least about: 1 time per day, 2 times per day, 3 times per day, 4 times per day, 5 times per day, 6 times per day or more than 6 times per day. In some cases, administering can be performed daily, weekly, monthly, or as needed. In some embodiments, administering can be conducted one, twice, three, or four times per day. In some cases, administration can be performed by a subject (e.g., the patient), a health care provider, or both.

Administration or application of a composition disclosed herein can be performed for a treatment duration of at least about, or equal to: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 300, 400, 500, 600, 700, 800, 900, or 1000 days consecutive or nonconsecutive days. In some cases, administration or application of a composition disclosed herein can be performed as needed. For example, administration or application of a composition disclosed herein can be administered when a subject has overdosed on an opioid (or a salt thereof) or is taking an opioid or a salt thereof. In some cases, a treatment duration can be from about 1 to about 30 days, from about 2 to about 30 days, from about 3 to about 30 days, from about 4 to about 30 days, from about 5 to about 30 days, from about 6 to about 30 days, from about 7 to about 30 days, from about 8 to about 30 days, from about 9 to about 30 days, from about 10 to about 30 days, from about 11 to about 30 days, from about 12 to about 30 days, from about 13 to about 30 days, from about 14 to about 30 days, from about 15 to about 30 days, from about 16 to about 30 days, from about 17 to about 30 days, from about 18 to about 30 days, from about 19 to about 30 days, from about 20 to about 30 days, from about 21 to about 30 days, from about 22 to about 30 days, from about 23 to about 30 days, from about 24 to about 30 days, from about 25 to about 30 days, from about 26 to about 30 days, from about 27 to about 30 days, from about 28 to about 30 days, from about 29 to about 30 days, from about 1 to about 90 days, from about 30 day to about 90 days, from about 60 days to about 90 days, from about 30 days to about 180 days, or from about 90 days to about 190 days.

Administration or application of a composition disclosed herein can be performed for a treatment duration of at least about 1 week, at least about 1 month, at least about 1 year, at least about 2 years, at least about 3 years, at least about 4 years, at least about 5 years, at least about 6 years, at least about 7 years, at least about 8 years, at least about 9 years, at least about 10 years, at least about 15 years, at least about 20 years, or for life. Administration can be performed repeatedly over a lifetime of a subject, such as once a month or once a year for the lifetime of a subject. Administration can be performed repeatedly over a substantial portion of a subject's life, such as once a day, once a week, once a month or once a year for at least about 1 year, 5 years, 10 years, 15 years, 20 years, 25 years, 30 years, or more.

Administration or application of a composition disclosed herein can be performed at least 1, 2, 3, 4, 5, 6, 7, 8, 9, 10, 11, 12, 13, 14, 15, 16, 17, 18, 19, 20, 21, 22, 23, or 24 times in a 24-hour period. In some cases, administration or application of a composition disclosed herein can be performed continuously throughout a 24-hour period, for example, when an implant can be used for administration. In some cases, administration or application of composition disclosed herein can be performed at least: 1, 2, 3, 4, 5, 6, 7, 8, 9, 10, 11, 12, 13, 14, 15, 16, 17, 18, 19, 20, or 21 times a week. In some cases, administration or application of composition disclosed herein can be performed at least: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, or more than 90 times a month. In some cases, a composition can be administered as a single dose or as divided doses. In some cases, the compositions described herein can be administered at a first time point and a second time point. In some cases, a composition can be administered such that a first administration can be administered before the other with a difference in administration time of about: 1 hour, 2 hours, 4 hours, 8 hours, 12 hours, 16 hours, 20 hours, 1 day, 2 days, 4 days, 7 days, 2 weeks, 4 weeks, 2 months, 3 months, 4 months, 5 months, 6 months, 7 months, 8 months, 9 months, 10 months, 11 months, 1 year or more.

In some embodiments, administering can be performed for about: 1 day to about 8 days, 1 week to about 5 weeks, 1 month to about 12 months, 1 year to about 3 years, 3 years to about 10 years, 10 years to about 50 years, 25 years to about 100 years, or 50 years to about 130 years. In some embodiments, administering can be performed prior to the administering of an opioid or a salt thereof. In some embodiments, administering can be performed after the administration of an opioid or a salt thereof. In some embodiments, administering can be performed during the administration of an opioid or a salt thereof.

In some embodiments, the composition can be administered as needed, or for: one day, two days, three days, four days, five days, six days, a week, two weeks, three weeks, a month, two months, three months, four months, five months, six months, seven months, eight months, nine months, ten months, eleven months, a year, two years, or chronically.

In some cases, the peptide or the derivative thereof or the salt of any of these can be administered in a pharmaceutical composition, which can be in unit dose form. In some instances, the amount of the peptide, or the derivative thereof, or the salt of any of these can be dosed in an amount ranging from about 0.0001 mg/kg of body weight of the subject to about 1000 g/kg of body weight of the subject. In some cases, the dosage of a peptide per subject body weight, a derivative thereof, or salt of any of these, can be equal to, less than about, or more than about: 0.0001 grams (g)/kilogram (kg), 0.001 g/kg, 0.01 g/kg, 0.1 g/kg, 0.2 g/kg, 0.3 g/kg, 0.4 g/kg, 0.5 g/kg, 0.6 g/kg, 0.7 g/kg, 0.8 g/kg, 0.9 g/kg, 1 g/kg, 2 g/kg, 3 g/kg, 4 g/kg, 5 g/kg, 6 g/kg, 7 g/kg, 8 g/kg, 9 g/kg, 10 g/kg, 15 g/kg, 20 g/kg, 25 g/kg, 30 g/kg, 35 g/kg, 40 g/kg, 45 g/kg, 50 g/kg, 55 g/kg, 60 g/kg, 65 g/kg, 70 g/kg, 75 g/kg, 80 g/kg, 85 g/kg, 90 g/kg, 95 g/kg, 100 g/kg, 200 g/kg, 300 g/kg, 400 g/kg, 500 g/kg, 600 g/kg, 700 g/kg, 800 g/kg, 900 g/kg, or 1000 g/kg of subject body weight.

In some instances, the amount of a peptide, derivative thereof, or salt of any of these, which can be a pharmaceutically acceptable salt, that is dosed to the patient can range from 0.00001 mg to 1000 g. In some cases, an amount of a peptide, a derivative thereof, or salt of any of these in a composition can range from about 0.005 mg to about 1000 mg. In some cases, an amount of a peptide, a derivative thereof, or salt of any of these in a composition can be equal to, less than about, or more than about: 0.0001 g, 0.001 g, 0.01 g, 0.1 g, 1 g, 2 g, 3 g, 4 g, 5 g, 6 g, 7 g, 8 g, 9 g, 10 g, 15 g, 20 g, 25 g, 30 g, 35 g, 40 g, 45 g, 50 g, 55 g, 60 g, 65 g, 70 g, 75 g, 80 g, 85 g, 90 g, 95 g, 100 g, 200 g, 300 g, 400 g, 500 g, 600 g, 700 g, 800 g, 900 g, or 1000 g. In some cases, an amount of a peptide, a derivative thereof, or salt of any of these in a composition can be equal to, less than about, or more than about: 0.0001 mg, 0.001 mg, 0.01 mg, 0.1 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 200 mg, 300 mg, 400 mg, 500 mg, 600 mg, 700 mg, 800 mg, 900 mg, or 1000 mg. In some cases, an amount of a peptide, a derivative thereof, or salt of any of these can range from about: 0.0001 mg to about 1 mg, 0.01 mg to about 1 mg, 0.1 mg to about 1 mg, 1 mg to about 10 mg, 10 mg to about 100 mg, or 100 mg to about 1000 mg. In some cases, an amount of a peptide, a derivative thereof, or salt of any of these in a composition can range from about: 0.0001 g to about 1 g, 0.01 g to about 1 g, 0.1 g to about 1 g, 1 g to about 10 g, 10 g to about 100 g, or 100 g to about 1000 g.

In some instances, a composition such as a pharmaceutical composition may be in the form of a capsule, a tablet, a gummy, an oil, a liquid, a tincture, a lotion, a cream, a balm, a candy, a syrup, a food, a drink, an oil, a suppository, or a liquid for injection. In some cases, a composition can be in the form of an intra venous liquid for injection, an intra muscular liquid for injection, or subcutaneous liquid for injection.

### Diagnoses

In some embodiments, a method can further comprise diagnosing a subject as having the disease or condition. In some cases, a disease or condition can be an opioid-induced respiratory depression and/or opioid use disorder. In some embodiments, a diagnosing can comprise employing an in vitro diagnostic. In some embodiments, the in vitro diagnostic can be a companion diagnostic.

In some embodiments, a diagnosis can comprise a physical examination (e.g., a respiratory rate), a blood oxygen level, a radiological image (e.g., an imaging diagnostic), a blood, body fluid or tissue test, a respiratory rate, an antibody test, or any combination thereof. In some cases, a diagnosis can comprise determining the presence or absence of a diagnostic analyte. The diagnostic analyte can be a cytokine, such as a proinflammatory cytokine or a chemokine, or their receptors. In some cases, a diagnostic analyte can be the presence of an opioid in a biological sample.

In some embodiments, a diagnosis can comprise a radiological image and the radiological image can comprise: a computed tomography (CT) image, an X-Ray image, a magnetic resonance image (MRI), an ultrasound image, or any combination thereof. In some cases, a diagnosis can comprise detecting the presence of respiratory depression with an in vitro or in vivo assay.

In some aspects, a method may further comprise diagnosing a subject as having the disease. In some aspects, a diagnosing may comprise employing a physical examination, such as determining a respiratory rate of a subject. In some aspects, a diagnosing may comprise employing an in vitro diagnostic. In some aspects, a diagnosing may comprise employing an in vivo diagnostic In some aspects, the in vitro diagnostic may be a companion diagnostic.

### Kits

Also disclosed herein are kits comprising the pharmaceutical composition contained at least in part in packaging (e.g., a container). Also disclosed herein are methods of making kits comprising a pharmaceutical composition contained at least in part in packaging. In some cases, kits can comprise a container, such as a metal container, a plastic container, a glass container, or any suitable container.

### Methods of Treatment

Also disclosed herein are methods of treating a disease or condition comprising administering a therapeutically effective amount of a pharmaceutical composition, such as a peptide disclosed herein. For example, a peptide can comprise RAP-103, DAPTA, a salt of any of these, or any combination thereof. In some cases, provided herein are compositions and formulations for use using any of the methods described. For example, a composition and formulation can be used in treating a disease or condition such as an opioid-induced respiratory depression.

In some embodiments, the disease or condition is an opioid-induced respiratory depression. In some cases, opioid-induced respiratory depression can be induced by an opioid via activation of u-opioid receptors in the central nervous system. In some cases, activation of μ-opioid receptors can include activation in the pre-Bötzinger complex, which is a respiratory rhythm generating area located in the pons. In some cases, the disease or condition can comprise an opioid physical dependence, opioid use disorder, an opioid addiction, an opioid dependence, an opioid side effect, or any combination thereof. In some cases, an opioid side effect can comprise a drowsiness, a confusion, a euphoria, a diarrhea, poor motor coordination, a slowed thinking, an impaired judgement, an impaired problem-solving, an emotional swing, an irritability, a nausea, a constipation, or any combination thereof. In some cases, a composition herein can reduce an adverse opioid effect such as respiratory depression, physical dependence, reinforcing efficacy, or any combination thereof.

In some cases, an opioid related disorder herein, such as opioid-induced respiratory depression can be caused by an opioid such as, an oxycodone, an oxymorphone, a hydrocodone, a hydromorphone, a fentanyl, a morphine, a codeine, an opium, a methadone, a tramadol, a buprenorphine, a heroin, a meperidine, a dextromethorphan, a dextropropoxyphene, a loperamide, an oxymorphone, a carfentanil, a tapentadol, a salt of any of these, a derivative of any of these, or any combination thereof. In some cases, an opioid can comprise a synthetic opioid, a natural opioid, a salt of any of these, or a mixture thereof. In some instances, a subject may be administered an opioid such as an opioid described above prior to, after, or during treatment with a peptide or salt thereof.

In some cases, a method of treatment can comprise increasing oxygen saturation levels in a subject who has taken an opioid or a salt thereof. In some cases, a method of treatment can comprise reducing or increasing a respiratory rate in a subject who has taken an opioid or a salt thereof. In some cases, a method of treatment can comprise maintaining a respiratory rate in a subject who has taken an opioid or a salt thereof. In some cases, administration of a peptide herein can be used to increase oxygen saturation levels in a subject who has taken an opioid or a salt thereof. In some cases, administration of a peptide herein can be used to reduce, increase, or maintain a respiratory rate in a subject who has taken an opioid or a salt thereof.

In some embodiments, a subject who has received a composition (e.g., a peptide or a salt thereof) herein can have an increased oxygen saturation level (e.g., blood oxygen level). In some cases, a pulse oximeter can be used to measure an oxygen saturation (SpO₂) level. For example, a subject who has received a composition herein can have a blood oxygen level of more than or equal to about: 92%, 93%, 94%, 95%, 96%, 97%, 98%, or 99% as measured by a pulse oximeter. In some cases, a subject who has received a composition herein can have a blood oxygen level of about: 92% to 100%, 92% to 97%, 93% to about 98%, 94% to about 99%, 95% to about 99%, or 95% to about 98% as measured by a pulse oximeter. In some cases, the subject who received a composition may have taken opioids prior to receiving the composition. In some cases, the subject may have taken opioids after receiving the composition. In some cases, the subject who received a composition may not have taken opioids prior to receiving the composition.

In some embodiments, a subject who has received a composition (e.g., a peptide or a salt thereof) herein can have an increased oxygen saturation level (as measured by a pulse oximeter) of more than or equal to about: 2%, 3%, 4%, 5%, 6%, 7%, 8%, 9%, 10%, 11%, or 12% as compared to a subject who was not administered the composition. In some cases, a subject may have taken opioids prior to receiving the composition. In some cases, the subject may have taken opioids after receiving the composition. In some cases, the increase in oxygen saturation level in a subject who received a composition herein can occur after about: 1 min to about 60 minutes, 30 seconds to about 10 minutes, 10 minutes to about 30 minutes, or 20 minutes to about 60 minutes. In some cases, the increase in oxygen saturation level in a subject who received a composition herein can occur after about: 10 seconds, 20 seconds, 30 seconds, 40 seconds, 50 seconds, 60 seconds, 1 min, 2 min, 3 min, 4 min, 5 min, 6 min, 7 min, 8 min, 9 min, 10 min, 11 min, 12 min, 13 min, 14 min, 15 min, 16 min, 17 min, 18 min, 19 min, 20 min, 21 min, 22 min, 23 min, 24 min, 25 min, 26 min, 27 min, 28 min, 29 min, 30 min, 31 min, 32 min, 33 min, 34 min, 35 min, 36 min, 37 min, 38 min, 39 min, 40 min, 41 min, 42 min, 43 min, 44 min, 45 min, 46 min, 47 min, 48 min, 49 min, 50 min, 51 min, 52 min, 53 min, 54 min, 55 min, 56 min, 57 min, 58 min, 59 min, or 60 min.

In some embodiments, a subject who has received a composition (e.g., a peptide or a salt thereof) herein can have a reduced respiratory rate. In some cases, a subject who has opioid-induced repertory depression can have an increased respiratory rate due to hypoxia. In some cases, a subject who has received a composition can have a reduced respiratory rate of more than, less than, or equal to about: 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% as compared to a subject who did not receive the composition as measured by cumulative respiratory rate in 60 minutes. In some cases, a subject who has received a composition can have a reduced respiratory rate of about: 8% to about 30%, 10% to about 25%, 15% to about 25%, 18% to about 24%, 20% to about 25%, 15% to about 20% or 20% to about 30% as compared to a subject who did not receive the composition as measured by cumulative respiratory rate in 60 minutes. In some cases, a subject may have taken opioids prior to receiving the composition. In some cases, the subject may have taken opioids after receiving the composition. In some cases, a subject who has received a composition can have a reduced respiratory rate of more than, less than, or equal to about: 10%, 15%, 20% or 25% as compared to a subject who did not receive the composition as measured by cumulative respiratory rate in 60 minutes.

In some embodiments, a composition herein can increase, decrease, or maintain OPRM1 (mu-opioid receptor) and TH (tyrosine hydroxylase) levels in the ventral tegmental area (VTA), the nucleus accumbens (NAC), the prefrontal cortex (PFC) or any combination thereof. In some cases, OPRM1 levels in the VTA are decreased after administration of a composition herein. In some cases, OPRM1 levels are decreased in the VTA by about: 20% to about 50%, 25% to about 45%, or 30% to about 40% as compared to OPRM1 levels in a subject who did not receive the composition. In some cases, OPRM1 levels are decreased in the VTA by more than, less than, or equal to about: 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%, as compared to OPRM1 levels in a subject who did not receive the composition. In some cases, OPRM1 levels are increased in the PFC after administration of a composition herein by about: 50% to about 70%, 55% to about 65% or 60% to about 70% as compared to OPRM1 levels in a subject who did not receive the composition. In some cases, OPRM1 levels are increased in the PFC after administration of a composition herein by more than, less than, or equal to about: 50%, 51%, 52%, 53%, 54%, 55%, 56%, 57%, 58%, 59%, 60%, 61%, 62%, 63%, 64%, 65%, 66%, 67%, 68%, 69%, or 70% as compared to OPRM1 levels in a subject who did not receive the composition. In some cases, TH levels are increased in the PFC after administration of a composition herein by about: 20% to about 45%, 30% to about 40% or 25% to about 35% as compared to TH levels in a subject who did not receive the composition. In some cases, TH levels are increased in the PFC after administration of a composition herein by more than, less than, or equal to about: 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, or 45%, as compared to TH levels in a subject who did not receive the composition. In some cases, a subject may have taken opioids prior to receiving the composition. In some cases, the subject may have taken opioids after receiving the composition.

In some embodiments, compositions and formulations herein can treat neuropathic pain. In some cases, the compositions herein can be used to downregulate phagocytosis, immune cell migration, cell membrane ruffling, or any combination thereof.

In some embodiments, compositions and formulations can be used to inhibit chemokine ligands from binding to CCR2, CCR5, CCR8, and/or CXCR4. In some instances, opioid-induce respiratory depression can be treated by inhibiting chemokine ligands from binding to CCR2, CCR5, CCR8, and/or CXCR4. In some cases, compositions and formulations can lower cytokines including IL-1, IL-6, IL-8 and TNF-alpha as compared to the a cytokine level before treatment.

In some embodiments, compositions and formulations can be used to treat an emotional instability, a depression, an anxiety, a panic attack, a suicidal ideation, or any combination thereof. In some cases, an emotional instability can comprise a range of emotions such as rage, sorrow, shame, panic, terror, emptiness, loneliness, or any combination thereof. In some cases, emotional instability can comprise an emotionally unstable personality disorder. In some cases, a depression can comprise a major depressive disorder, a bipolar depression, a perinatal depression, a postpartum depression, a persistent depressive disorder, a premenstrual dysphoric disorder, a psychotic depression, a seasonal affective disorder, atypical depression, or any combination thereof. In some cases, an anxiety can comprise a generalized anxiety disorder, a obsessive-compulsive disorder, a panic disorder, a post-traumatic stress disorder, a social phobia, an agoraphobia, a phobia, a social anxiety disorder, a specific phobia, or any combination thereof. In some cases, a panic attack can comprise an expected panic attack, or an unexpected panic attack. In some cases, suicidal ideation can comprise an active suicidal ideation or a passive suicidal ideation. In some cases, a composition herein can be used to treat a paranoia.

### Co-Therapies

In some embodiments, a method may further comprise administering a second therapy to the subject. In some cases, a pharmaceutical composition can further comprise a second therapy. In some cases, the administration of a second therapy is separate from the administration of the first pharmaceutical composition. In some aspects, a second therapy may comprise a buprenorphine, a methadone, a naltrexone, an extended release naltrexone, a naloxone, a salt of any of these, or any combination thereof. In some cases, a naloxone can comprise intravenous naloxone, intramuscular naloxone, subcutaneous naloxone, intranasal naloxone, a salt of any of these, or any combination thereof.

In some embodiments, a second therapy can comprise a therapy, a psychotherapy, a speech therapy, a physical therapy, an occupational therapy, or any combination thereof.

In some embodiments, a method can comprise administering 1, 2, 3, 4, 5, 6, or more than 6 additional therapies. In some instances, any second therapy disclosed herein can be used as an additional therapy. In some cases, a second therapy or additional therapy can be administered concurrently or consecutively with a peptide disclosed herein.

In some aspects, the second therapy or additional may be administered as needed, or for: one day, two days, three days, four days, five days, six days, a week, two weeks, three weeks, a month, two months, three months, four months, five months, six months, seven months, eight months, nine months, ten months, eleven months, a year, or chronically. In some cases, a second therapy can be administered by any method of administration described herein.

In some aspects, the second therapy or additional therapy may be administered so that the active ingredient or the pharmaceutically acceptable salt thereof in the unit dose ranges from about: 500 μg (micrograms) to about 1000 mg, 10 μg to about 50 μg, 40 μg to about 90 μg, 80 μg to about 120 μg, 100 μg to about 150 μg, 140 μg to about 190 μg, 150 μg to about 220 μg, 200 μg to about 250 μg, 240 μg to about 300 μg, 290 μg to about 350 μg, 340 μg to about 410 μg, 400 μg to about 450 μg, 440 μg to about 500 μg, 500 μg to about 700 μg, 600 μg to about 900 μg, 800 μg to about 1 mg, 1 mg to about 5 mg, 1 mg to about 10 mg, 5 mg to about 15 mg, 12 mg to about 25 mg, 20 mg to about 50 mg, 40 mg to about 80 mg, 70 mg to about 100 mg, 90 mg to about 150 mg, 125 mg to about 250 mg, 200 mg to about 500 mg, 400 mg to about 750 mg, 700 mg to about 900 mg, or from about 850 mg to about 1000 mg. In some cases, the unit dose range may be equal to, less than about, or more than about: 10 μg, 25 μg, 50 μg, 75 μg, 100 μg, 150 μg, 200 ug, 220 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, or 25 mg. In some cases, the unit dose range may be less than about: 10 ug, 25 μg, 50 μg, 75 μg, 100 μg, 150 μg, 200 μg, 220 μg, 250 μg, 300 μg, 350 μg, 400 μg, 450μ, 500μ, 550μ, 600μ, 650μ, 700μ, 750μ, 800μ, 850μ, 900μ, 950μ, 1000μ, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, or 25 mg.

### EXAMPLES

The following examples are included for illustrative purposes only and are not intended to limit the scope of the disclosure.

### Example 1: Use of D Containing Amino Acid Peptides to Treat Opioid-Induced Respiratory Depression

The effects of RAP-103 (a peptide having the following amino acid sequence from the N terminus to the C terminus: D-Thr-D-Thr-D-Asn-D-Tyr-D-Thr (SEQ ID NO: 1); where D indicates the amino acid is of the D stereochemical configuration, and Thr stands for threonine, Asn stands for asparagine, Tyr stands for tyrosine), a multi-chemokine receptor antagonist (CRA) small peptide, on respiratory depression and dependence in male rats was determined. Further the effects of opioid exposure on chemokine and mu-opioid receptor levels in mesolimbic substrates was assessed. Acute morphine (5 mg/kg, subcutaneous or SC) reduced oxygen saturation (SpO₂) and enhanced respiratory rate, and both effects were abolished by pretreatment with RAP-103 (1 mg/kg, intraperitoneal or IP). In rats exposed to chronic morphine (75 mg pellet×7 d), daily treatment with RAP-103 (1 mg/kg, IP) reduced naloxone-precipitated withdrawal responses (wet-dog shakes and eye blinking). For self-administration (SA) experiments, RAP-103 (1 mg/kg, IP) reduced heroin acquisition (0.1 mg/kg/inf) and heroin-reinforced breakpoints without affecting sucrose intake. Abstinence from chronic morphine exposure resulted in brain-region specific changes in chemokine receptor protein levels (CCR2 and CXCR4 were increased in the ventral tegmental area (VTA), and CCR2 and CCR5 were reduced in the nucleus accumbens (NAC). RAP-103 (1 mg/kg, IP) normalized morphine-induced chemokine receptor dysregulation in the NAC but not VTA. These results indicate that CRAs can function as biphasic function opioid modulators that enhance opioid analgesia but inhibit opioid dependence and respiratory depression. RAP-103 and related peptides such as DAPTA can be used to treat opioid-induced respiratory depression.

### Methods and Materials

### Subjects

Adult male Sprague Dawley (226-275 g) were purchased from Taconic Laboratories and maintained on a 12-hour/12-hour light/dark cycle with the lights off at 9:30 a.m. in a temperature- and humidity-controlled animal care facility. For the duration of the experiment, rats were housed two per cage and given unrestricted access to food and water at all times when in the home cage.

Morphine sulfate, morphine pellets (75 mg each), and heroin (3,6-Diacetylmorphine hydrochloride) were obtained from NIDA Drug Supply. Naloxone hydrochloride was purchased from Sigma-Aldrich (St. Louis, MO, USA). RAP-103, which is a pentapeptide orally active analog of Peptide T/“DAPTA”, was synthesized. RAP-103 was purified to >95% homogeneity and verified by high performance liquid chromatography isolation, amino acid analysis, and mass spectroscopy. All drugs were dissolved in 0.9% physiological saline.

### Effect of RAP-103 on Morphine-Induced Respiratory Depression

Oxygen saturation (SpO₂) and respiratory rate (RR) were measured using a MouseOX® Plus-pulse oximeter (mouse and rat pulse oximeter, Starr Life Sciences Corp., Oakmont, PA, USA) in freely moving rats. Rats were acclimated individually in observation boxes for 30 min. Then, a collar with a sensor was placed to the neck of the rats and without any recording, rats were acclimated for another 30 min. To test whether pretreatment with RAP-103 would have an effect on morphine-induced respiratory depression, rats were pretreated with either saline or RAP-103 (1 or 3 mg/kg, IP). Thirty min later, rats were injected with either saline or morphine (5 mg/kg, SC). The dose of morphine was based on previous studies showing that 5 mg/kg reduces oxygen saturation and enhances respiratory rate. Following the injection of morphine or saline, SpO₂ and RR were recorded for 60 min. Rats were assigned randomly in one of the 6 groups (n=6-7): vehicle+saline; vehicle+morphine (5 mg/kg); RAP-103 (1 mg/kg)+saline; RAP-103 (3 mg/kg)+saline; RAP-103 (1 mg/kg)+morphine; or RAP-103 (3 mg/kg)+morphine. At the end of the 60-min recording, rats were euthanized.

### Effect of RAP-103 on Development of Morphine Physical Dependence

Rats were implanted (SC, flank area) with 75 mg morphine pellets that remained intact for 7 days. Beginning on the day of pellet implantation, rats received daily injections of saline or RAP-103 (0.001, 0.1 or 1 mg/kg, IP, delivered in a ml/kg volume) throughout chronic morphine exposure. On the test day, rats were transported to the testing room and allowed to acclimate for 60 min before naloxone hydrochloride (10 mg/kg, IP, delivered in a 1 ml/kg volume) was injected to precipitate withdrawal signs. Immediately after naloxone administration, rats were placed into individual observation boxes and observed for 60 min. A modified version of the Gellet-Holtzman scale was used to grade withdrawal signs. Wet dog shakes, teeth chattering, eye blinks, and paw tremor were evaluated as the number of qualitatively observed occurrences over the 60-min test time.

### Jugular Catheterization Surgeries for Self-Administration (SA) Experiments

Rats were anesthetized using vaporized isoflurane delivered via nebulizer. An indwelling silastic catheter was threaded subcutaneously over the shoulder blade, inserted in the jugular vein and sutured in place. The catheter routed to a cotton back mount platform that was sutured below the skin between the shoulder blades. Post-operation, catheters were flushed daily with 0.2 ml of timentin (0.93 mg/mL) dissolved in heparinized saline and covered with magnetic aluminum caps.

### Effects of RAP-103 on Heroin Intravenous Self-Administration (SA)

Following 5 days of post-operative recovery, rats began daily intravenous self-administration of heroin in Med-Associates operant chambers for at least 10 d. The chambers were equipped with a sustained house light, an active response lever that delivered drug infusions and an inactive lever that had no drug-paired consequence. During IVSA, infusions of 0.1 mg/kg/inf heroin were rewarded to the animal after pressing the active lever. The volume for each infusion was 59 microliters delivered over 5 s. Each infusion was paired with the illumination of a cue light and was followed by a 20-s “time-out” period, during which the house light was off, and responses were not rewarded. Both active and inactive lever responses were recorded using Med-Associates software. Each session was 6.5 h in length and drug availability was intermittent, such that animals were exposed to 60-min drug delivery sessions as described above, with 5 min breaks in between each session where no drug, cue lights, or levers were available. Heroin SA included the following reward schedules: fixed ratio 1 (FR1), fixed ratio 3 (FR3), and progressive ratio (PR). RAP-103 (1 mg/kg, IP) or saline was injected 30 min before each daily heroin SA session.

Progressive ratio (PR) schedules of drug delivery are used to determine the ‘motivational’ state of an animal and drug reinforcing efficacy. A progressive ratio (PR) schedule requires the subject to perform an increasing number of lever presses for the next successive presentation of a reinforcer (e.g., heroin or sucrose). The maximal number of presses resulting in the final administration of a reinforcer is called ‘breaking point’ or ‘breakpoint’ (BP). BP is a dependent measure used to quantify the degree to which the subject will work to get the next administration of the drug which serves as a reinforcer in the self-administration paradigm. The BP magnitude can be considered to be proportional to the abuse liability of the drug and/or motivation to obtain it.

### Effects of RAP-103 on Sucrose Intravenous Self-Administration (SA)

Sucrose self-administrations sessions also took place in Med-Associates operant chambers equipped with a sucrose hopper, house light, active lever and inactive lever. Subjects were given access to self-administer sucrose pellets for 12-14 d for 2 h each day. A single sucrose pellet reward was delivered to subjects via a food-trough after pressing the active lever. Each pellet delivery was paired with the illumination of a cue light and was again followed by a 20-s “time-out” period. All responses were recorded using the Med-Associates software. Sucrose SA included the following reward schedules: fixed ratio 1 (FR1), fixed ratio 3 (FR3), and progressive ratio (PR). RAP-103 (1 mg/kg, IP) or saline was injected 30 min before each daily sucrose SA session.

Effects of RAP-103 and morphine exposure on chemokine and opioid receptor protein levels in the mesolimbic pathway

For 10 d, rats were given two injections during the dark cycle. One injection (RAP-103 [1 mg/kg] or saline) was delivered IP, and 5 min later the second injection (morphine [5 mg/kg] or saline) was given SC. Brain tissue was collected 24 h after the last morphine or saline injection for protein analysis of chemokine and opioid markers in the ventral tegmental area (VTA), nucleus accumbens (NAC) and prefrontal cortex (PFC).

For immunoblotting experiments, tissue lysates were obtained using RIPA buffer, and were then homogenized. Samples were centrifuged for 15 minutes at 12,000 g. Supernatant were transferred to new tubes. The samples were heated at 95 °C for 5 min, resolved by SDS-PAGE gel electrophoresis and transferred onto nitrocellulose membrane. After blocking with blocking buffer (Licor) for 1 hour, the membrane was incubated for overnight at 4 °C with the corresponding primary antibodies. The membranes were washed with TBS and then incubated with secondary antibodies for 2 hours at room temperature. The membranes were scanned by using Licor Odssey-2014. Immunoreactive bands were quantified by Image Studio (Licor Ver 2.0) and normalized to the bands of GAPDH. The data were then imported into GraphPad Prism 7 for gRhing and statistical analysis. Antibodies and materials used were: CCR2 polyclonal antibody, CXCR4 polyclonal antibody, CCR5 polyclonal antibody, mu-opioid receptor (OPRM1) polyclonal antibody, Tyrosine hydroxylase (TH) polyclonal antibody, and Blocking buffer, Licor.

### Statistical Analysis

Respiratory depression data were analyzed by two-way ANOVA (RAP-103 pretreatment×morphine treatment) followed by a Bonferroni's multiple comparison test. Physical dependence data were analyzed by a Student's t-test. Self-administration (SA) time-course data for heroin and sucrose were analyzed by two-way ANOVA (RAP-103 treatment x day) followed by a Bonferroni's multiple comparison test. SA PR challenge data were analyzed by a Student's t-test. Protein and mRNA data were analyzed by two-way ANOVA (RAP-103 pretreatment x morphine treatment) followed by a Newman-Keuls post-hoc analysis. In all cases, P<0.05 was considered statistically significant.

### Results

### RAP-103 Normalized Morphine-Induced Reduction in Oxygen Saturation (SpO2)

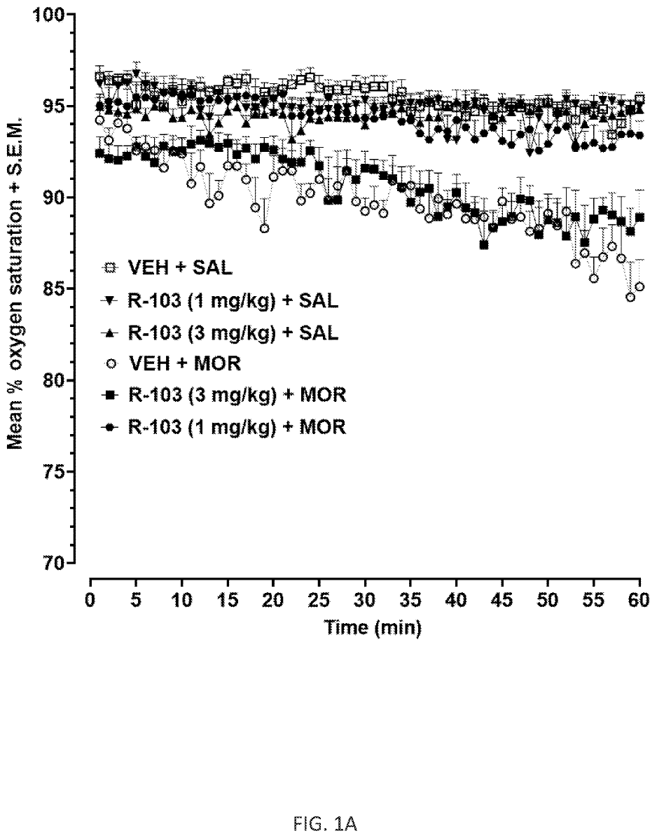

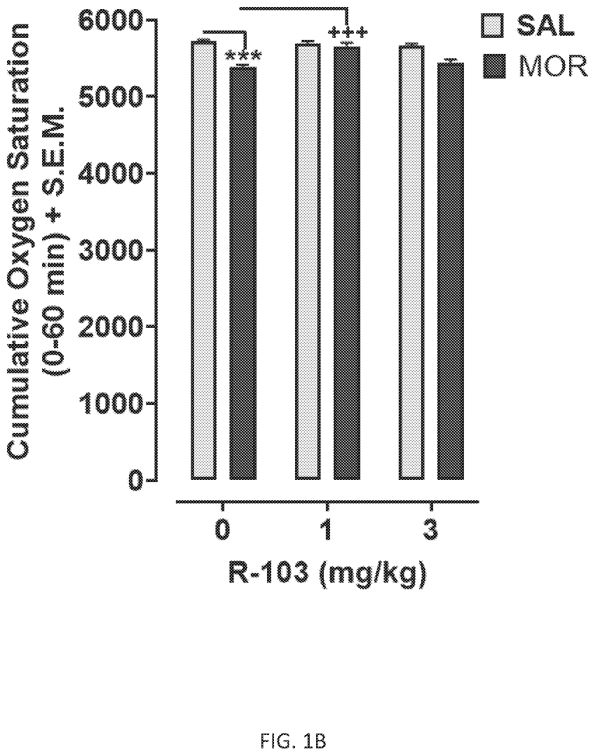

RAP-103 administered prior to morphine exposure (5 mg/kg) inhibited the reduction in peripheral capillary oxygen saturation (SpO₂) caused by morphine. For time-course data as shown in FIG. 1A, two-way ANOVA revealed effects of group [F(5,30)=19.75, P<0.0001] and time [F(1,30)=58.44, P<0.0001] and a significant interaction [F(295,1770)=1.994, P<0.0001]. Morphine decreased SpO₂ to under 90%, especially after 50 min, which is indicative of hypoxia. For cumulative SpO₂ (entire 60-min interval following morphine exposure) as shown in FIG. 1B, two-way ANOVA revealed effects of RAP-103 pretreatment [F(2,30)=9.617, P<0.0001] and morphine treatment [F(1,30)=58.44, P<0.0001] and a significant interaction [F(2,30)=10.54, P<0.0001]. In RAP-103-naïve rats (0 mg/kg RAP-103), post-hoc analysis revealed that treatment with morphine significantly reduced SpO₂ compared to treatment with saline (P<0.001). In rats treated with morphine (5 mg/kg), pretreatment with RAP-103 (1 mg/kg) normalized the morphine-induced reduction in cumulative SpO₂ (i.e., SpO₂ was lower in morphine-treated rats that did not receive RAP-103 pretreatment) (P<0.001). RAP-103 by itself did not produce effects on SpO₂ (i.e., SpO₂ in morphine-naïve rats injected with RAP-103 (1, 3 mg/kg) was not different from saline-injected rats) (P>0.05).

### RAP-103 Normalized Morphine-Induced Enhancement of Respiratory Rate

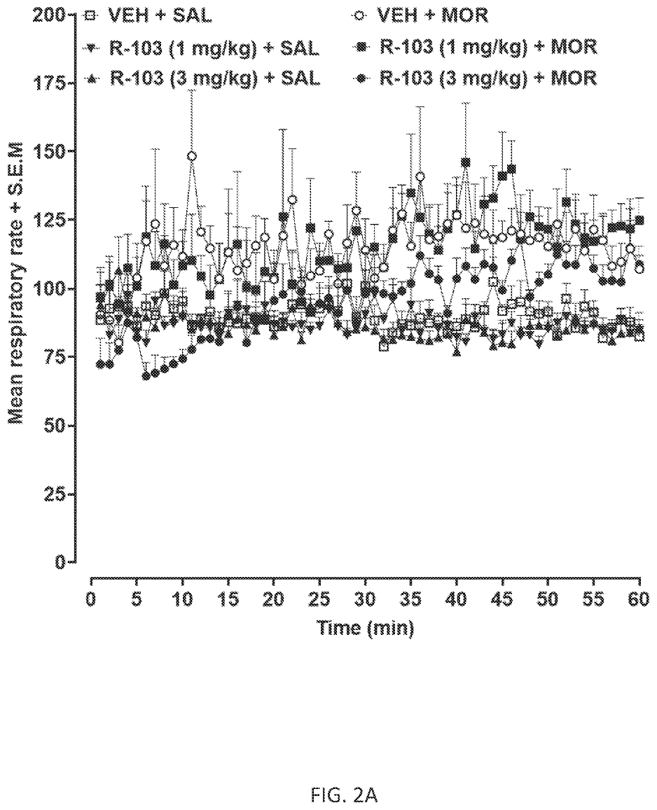

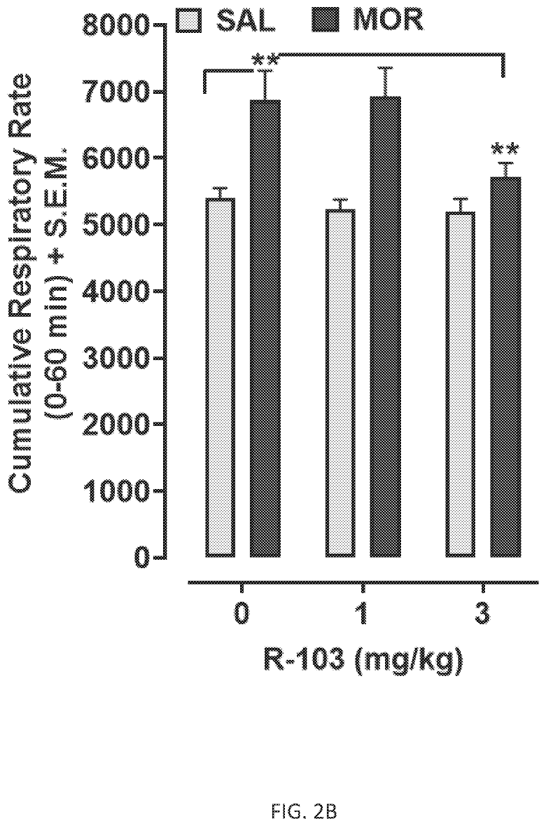

RAP-103 administered before morphine exposure (5 mg/kg) normalized the enhancement in respiratory rate caused by morphine. For time-course data as shown in FIG. 2A, two-way ANOVA revealed a group [F(5,30)=7.95, P<0.0001] effect and a significant interaction [F(295,1770)=1.54, P<0.0001]. For cumulative respiratory rate (entire 60-min interval following morphine exposure) as shown in FIG. 2B, two-way ANOVA revealed effects of RAP-103 pretreatment [F(2,30)=3.51, P<0.05] and morphine treatment [F(1,30)=27.84, P<0.0001]. In RAP-103-naïve rats (0 mg/kg RAP-103), post-hoc analysis revealed that treatment with morphine significantly increased respiratory rate (a compensation due to hypoxia) compared to treatment with saline (P<0.01). In rats treated with morphine (5 mg/kg), pretreatment with RAP-103 (3 mg/kg) normalized the morphine-induced increase in respiratory rate (i.e., respiratory rate was lower in morphine-treated rats that received RAP-103 pretreatment) (P<0.01). RAP-103 by itself did not produce effects on respiratory rate (e.g., respiratory rate in morphine-naïve rats injected with RAP-103 (1, 3 mg/kg) was not different from saline-injected rats) (P>0.05).

### RAP-103 Decreased Development of Morphine Physical Dependence

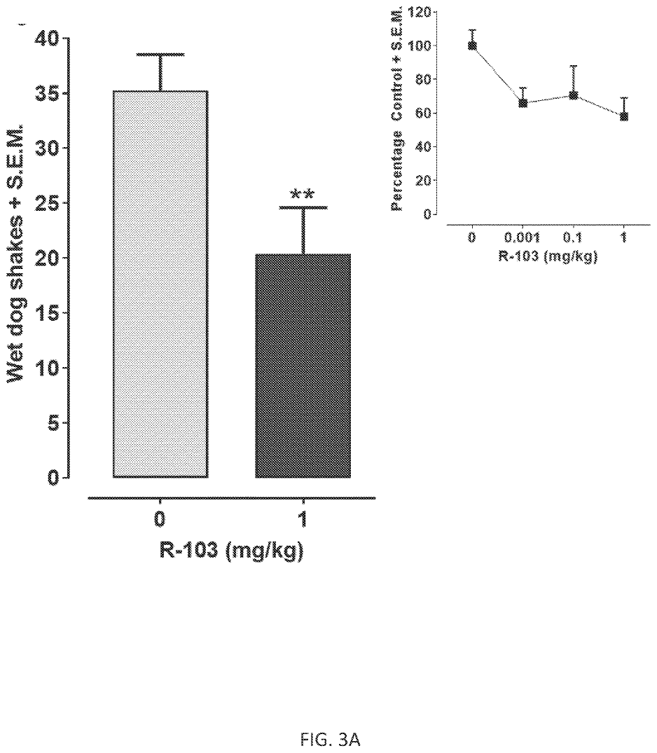

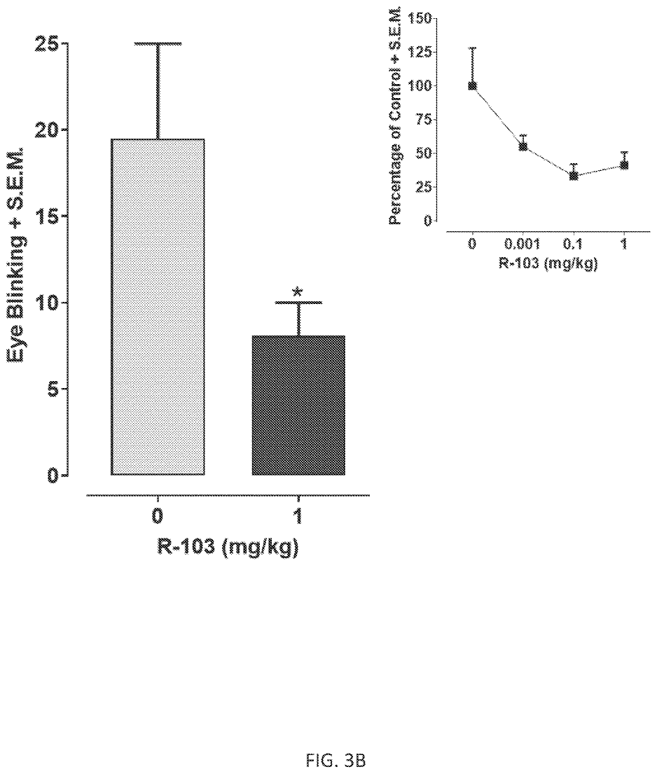

RAP-103 injected once daily during chronic morphine exposure (75 mg pellet, 7 d) reduced multiple metrices of naloxone-precipitated morphine withdrawal. For the endpoint of wet dog shakes as shown in FIG. 3A, a dose of 1 mg/kg RAP-103 significantly reduced the frequency of wet dog shakes (by approximately 43%) precipitated by naloxone challenge (P<0.01, Student's t-test). Treatment with lower doses of RAP-103 (0.001, 0.1 mg/kg) did not significantly impact naloxone-precipitated wet dog shakes, however a reduced trend was observed (P>0.05, Student's t-test). A dose of 1 mg/kg RAP-103 also significantly reduced the frequency of eye blinking (by approximately 59%) precipitated by naloxone challenge (P<0.05, Student's t-test) as shown in FIG. 3B. Treatment with lower doses of RAP-103 (0.001, 0.1 mg/kg) did not significantly impact naloxone-precipitated eye blinking (P>0.05, Student's t-test), however a reduced trend was observed. Two other withdrawal endpoints, teeth chattering and paw tremor, were not affected by any does of RAP-103.

### RAP-103 Decreased Heroin, but not Sucrose, Acquisition and Reinforcement

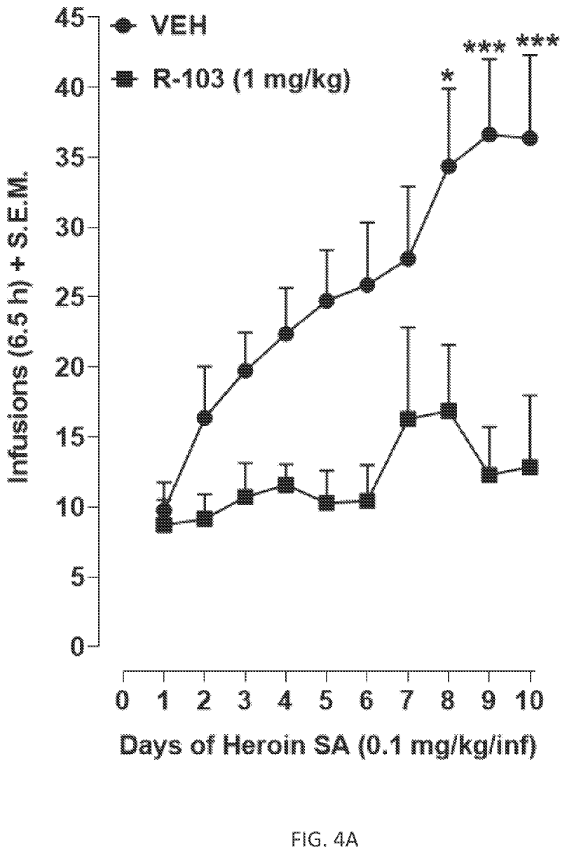

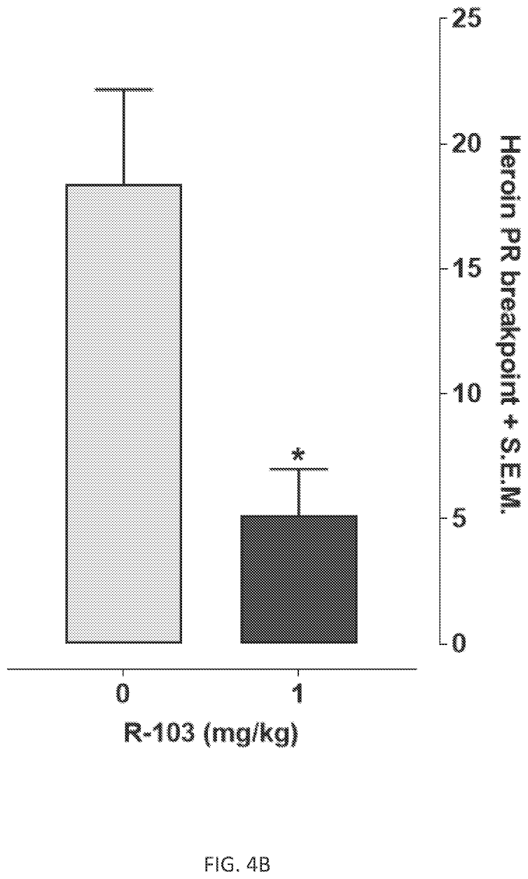

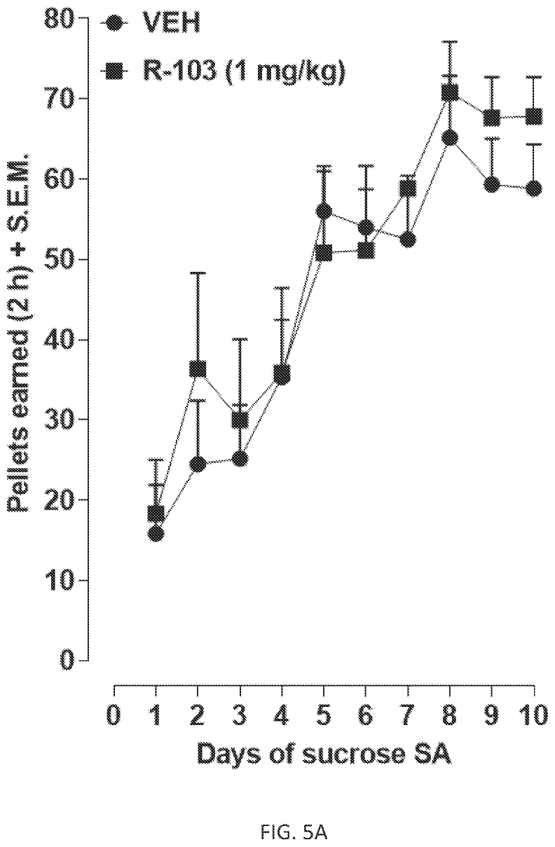

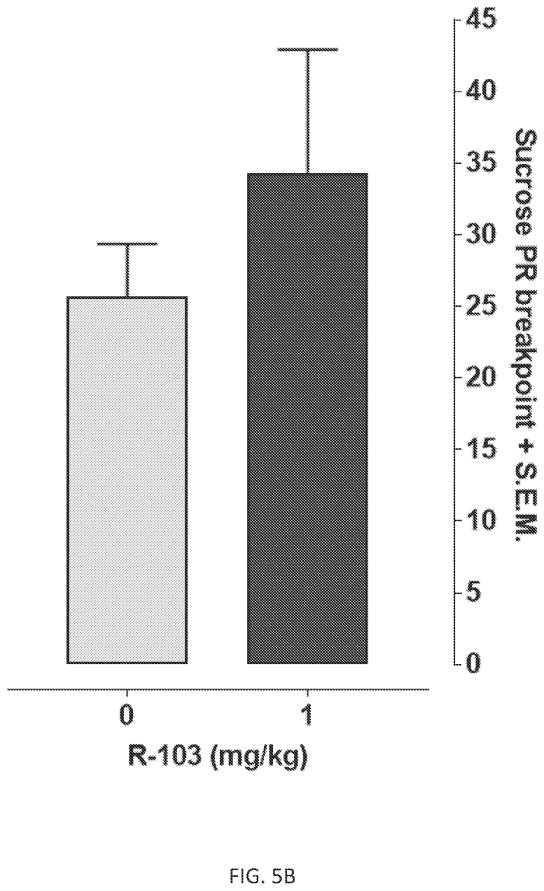

Rats underwent 10 d of heroin intravenous self-administration (SA) on a FR-1 reinforcement schedule, and a PR challenge at 0.1 mg/kg/infusion as shown in FIGS. 4A-4B. For time-course FR-1 data, as shown in FIG. 4A), two-way ANOVA revealed significant effects of treatment [F(1,130)=54.72, P<0.0001] and time [F(9,130)=3.59, P<0.0001]. Post-hoc analysis indicated that treatment with RAP-103 significantly reduced active lever responding on days 8 (P<0.05), 9 (P<0.001) and 10 (P<0.001) compared to saline-treated rats. For PR challenge, as shown in FIG. 4B, RAP-103-treated rats also exhibited significantly lower breakpoints in comparison to saline-treated control rats (P<0.05, Student's t-test). In separate experiments, rats underwent 10 d of sucrose SA on a FR-1 reinforcement schedule followed by a PR challenge as shown in FIGS. 5A-5B. For time-course data, as shown in FIG. 5A, sucrose-taking in rats treated with RAP-103 was not significantly different from rats treated with saline (P>0.05). In addition, for PR challenge, as shown in FIG. 5B, sucrose PR breakpoints were not significantly different in rats treated with RAP-103 or saline (P>0.05).

### RAP-103 Normalized Morphine-Induced Changes in Chemokine Receptors in NAC

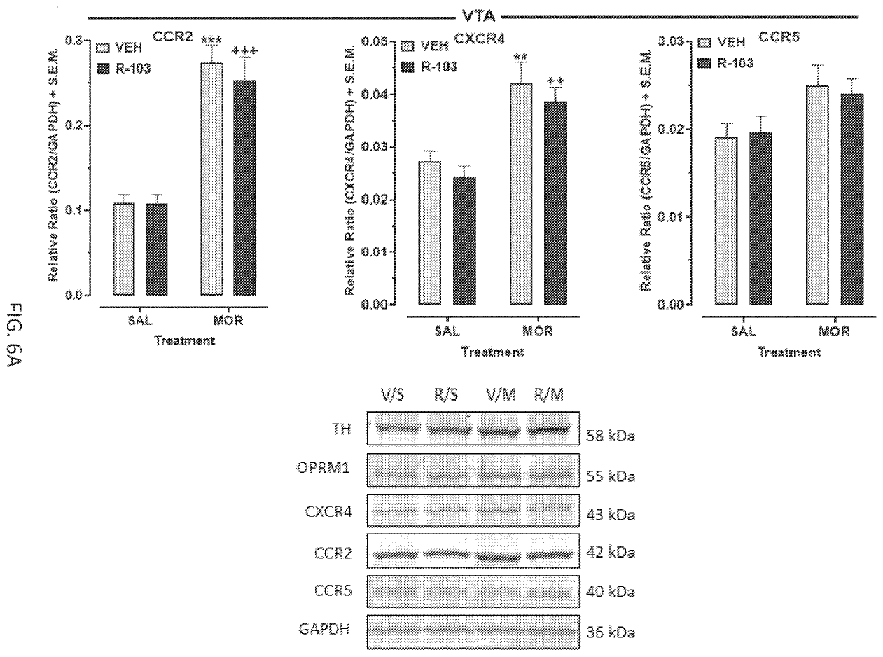

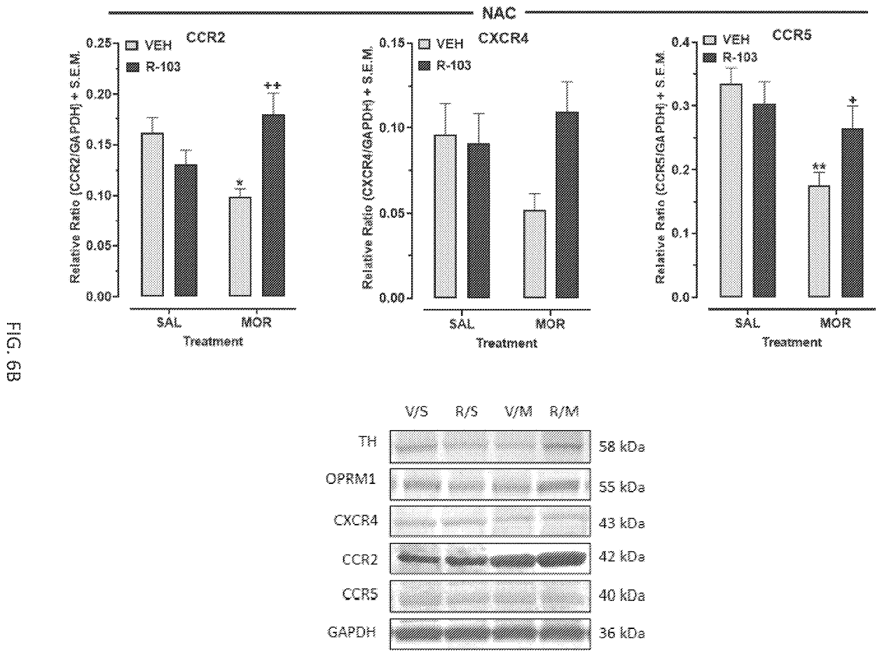

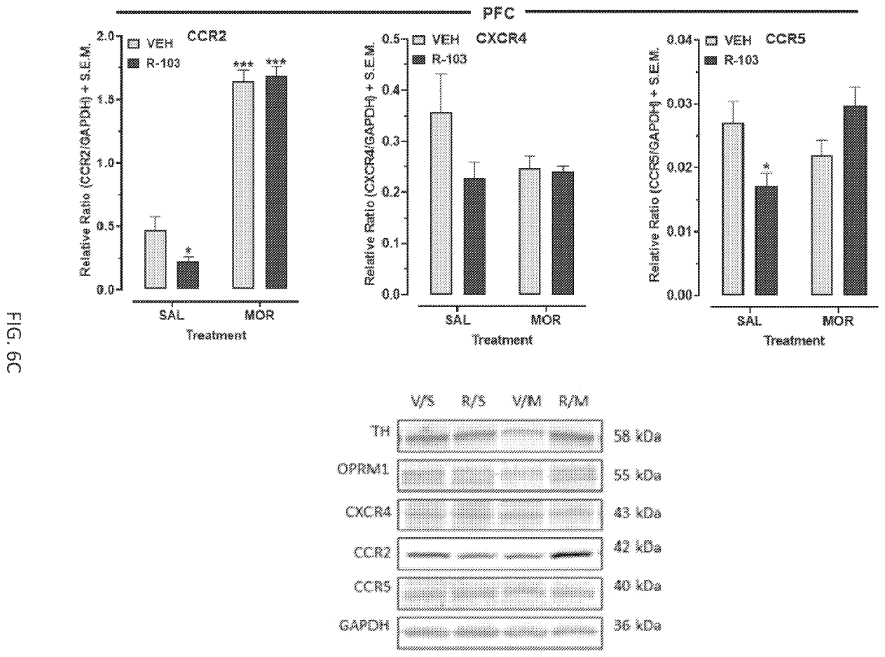

Chemokine receptor (CCR2, CXCR4 and CCR5) protein levels in mesocorticolimbic substrates were assessed 24 h after cessation of chronic (10 d) exposure to morphine and RAP-103, as shown in FIGS. 6A-6C. Results are separated and presented by brain region (FIG. 6A. ventral tegmental area (VTA); FIG. 6B, nucleus accumbens (NAC) and FIG. 6C, prefrontal cortex (PFC)). In the VTA, for CCR2, two-way ANOVA indicated a significant effect of morphine treatment [F(1,28)=73.77, P<0.0001] as shown in FIG. 6A. In comparison with drug-naïve rats, morphine treatment significantly enhanced CCR2 protein levels in rats pretreated with either vehicle or RAP-103 (P<0.001). For CXCR4 protein levels in the VTA, as shown in FIG. 6A, two-way ANOVA indicated a significant effect of morphine treatment [F(1,28)=27.25, P<0.0001]. In comparison with drug-naïve rats, morphine treatment significantly enhanced CXCR4 protein levels in rats pretreated with either vehicle or RAP-103 (P<0.001). However, protein levels of CCR5 in the VTA as shown in FIG. 6A), after 10 days morphine treatment did not significantly change. For rats receiving only RAP-103, levels of CCR2, CXCR4 or CCR5 were not significantly different in comparison to drug-naïve control rats (P>0.05), and RAP-103 did not alter morphine enhancement of CCR2 or CXCR4.

In contrast to the VTA, morphine treatment in the NCA caused a reduction in protein levels that was normalized by RAP-103 pretreatment. For CCR2, as shown in FIG. 6B, two-way ANOVA indicated an interaction between RAP-103 and morphine [F(1,27)=13.10, P<0.001]. In comparison with drug-naïve rats, morphine treatment significantly reduced CCR2 protein levels (P<0.05) and RAP-103 pretreatment normalized morphine-induced deficits in CCR2 levels (i.e., for morphine-treated rats, pretreatment with RAP-103 resulted in CCR2 protein levels that were significantly greater in comparison with vehicle pretreatment) (P<0.01). For CCR5, as shown in FIG. 6B, two-way ANOVA indicated a significant effect of morphine treatment [F(1,28)=11.52, P<0.01]. In comparison with drug-naïve rats, morphine treatment significantly reduced CCR5 protein levels (P<0.01). For morphine-treated rats as shown in FIG. 6B, RAP-103 pretreatment normalized morphine-induced deficits in CCR5 levels (i.e., rats treated with morphine that also received RAP-103 were significantly greater than morphine-treated rats that were naïve to RAP-103) (P<0.05). In comparison with drug-naïve controls, rats receiving only RAP-103 did not display significantly different levels of CCR2, CXCR4 or CCR5 (P>0.05).

In the PFC, for CCR2 as shown in FIG. 6C, two-way ANOVA indicated a significant effect of morphine treatment [F(1,28)=278,33, P<0.001]. In comparison with drug-naïve rats, morphine treatment significantly enhanced CCR2 protein levels in rats pretreated with either vehicle or RAP-103 (P<0.001). Also, in comparison with drug-naïve rats, rats receiving RAP-103 by itself displayed a reduction in CCR2 protein levels (P<0.05). For CCR5 as shown in FIG. 6C, two-way ANOVA indicated an interaction between RAP-103 and morphine [F(1,28)=10.84, P<0.01]. In comparison with drug-naïve rats, rats receiving RAP-103 displayed lower CCR5 levels (P<0.05).

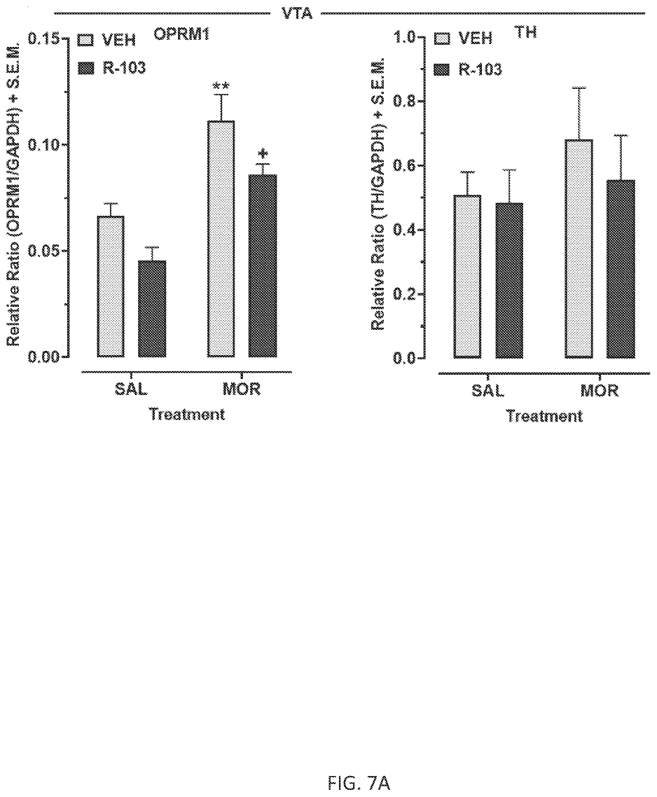

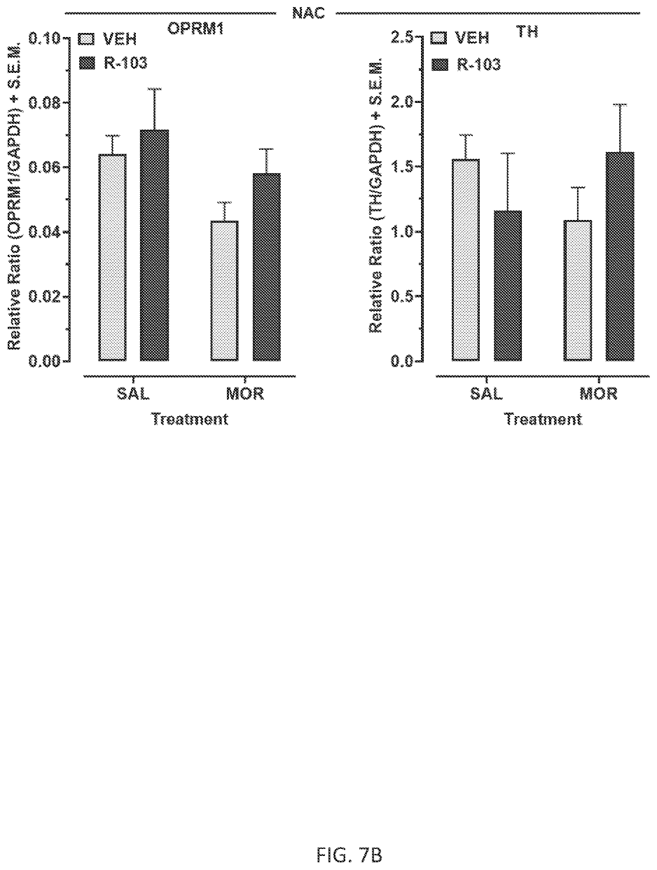

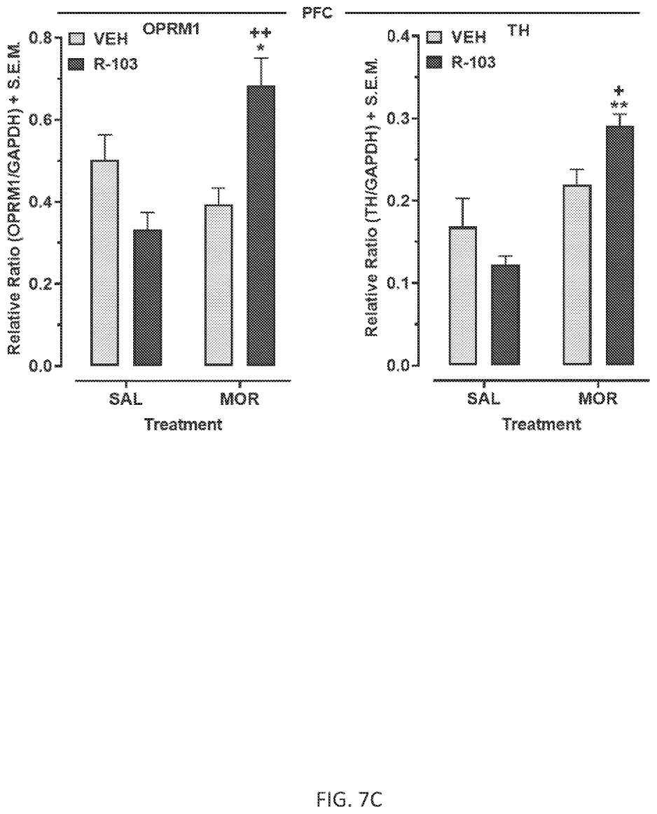

Mu-opioid (OPRM1) and tyrosine hydroxylase (TH) protein levels were also assessed 24 h following cessation of RAP-103/morphine exposure in each brain region as shown in FIGS. 7A-7C. In the VTA as shown in FIG. 7A, for OPRM1, two-way ANOVA indicated significant effects of RAP-103 pretreatment [F(1,27)=8.39, P<0.01] and morphine treatment [F(1,27)=28.41, P<0.001]. In comparison with drug-naïve rats, morphine treatment significantly increased OPRM1 protein levels (P<0.01). For morphine-treated rats, RAP-103 pretreatment normalized the morphine-induced enhancement in OPRM1 levels (i.e., for morphine-treated rats, pretreatment with RAP-103 resulted in CCR2 protein levels that were significantly lower in comparison vehicle pretreatment) (P<0.05) as shown in in FIG. 7A. In the PFC as shown in FIG. 7C, for OPRM1, two-way ANOVA indicated a significant effect of morphine treatment [F(1,28)=5.05, P<0.001] and a significant interaction [F(1,28)=18.21, P<0.001]. For rats receiving both morphine and RAP-103, levels of OPRM1 were significantly elevated in comparison to both drug-naïve rats (P<0.01) and rats that receiving only morphine (P<0.05). Significant impacts on TH protein levels were only detected in the PFC. Two-way ANOVA indicated a significant effect of morphine treatment [F(1,24)=26.96, P<0.001] and a significant interaction [F(1,24)=7.71, P<0.05] as shown in FIG. 7C. For rats receiving both morphine and RAP-103, TH levels were significantly elevated in comparison to both drug-naïve rats (P<0.05) and rats that receiving only morphine (P<0.01). FIG. 7B shows similar protein levels in the treatment group and vehicle group for OPRM1 and TH.

RAP-103 Normalized Morphine-Induced Enhancement of Astrocyte mRNA Levels in NAC

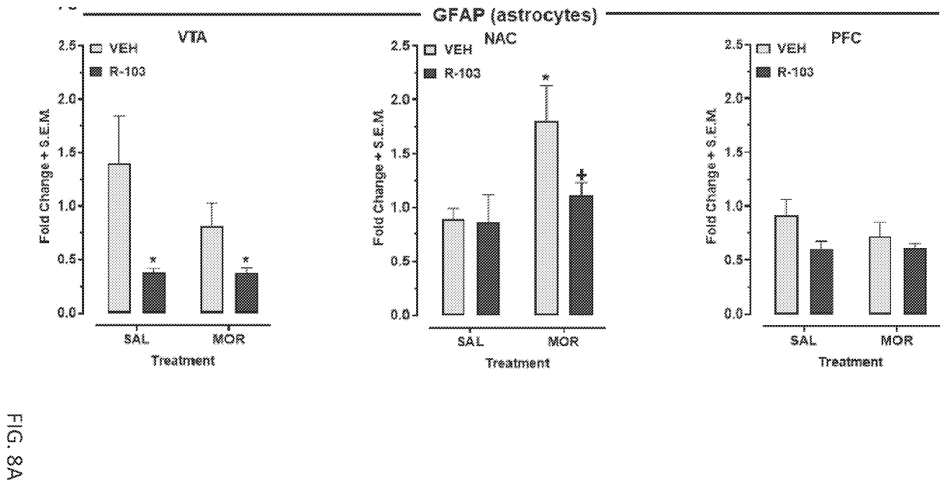

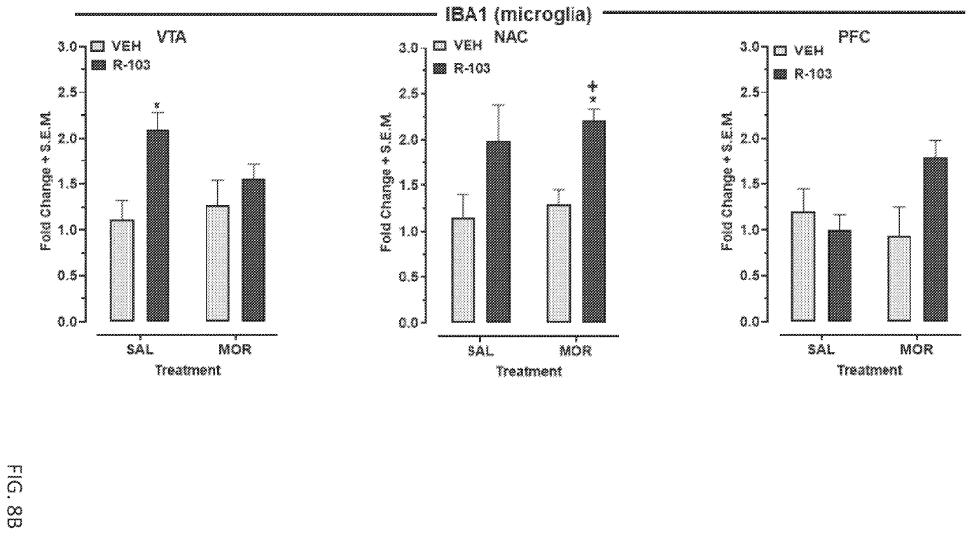

Astrocytic (GFAP) and microglial (IBA1) mRNA levels in mesocorticolimbic substrates were assessed 24 h after cessation of chronic (10 d) exposure to morphine and RAP-103 as shown in FIGS. 8A-8B. For GFAP gene expression as shown in FIG. 8A in the VTA, two-way ANOVA indicated a significant effect of RAP-103 pretreatment [F(1,27)=7.92, P<0.01]. In comparison with drug-naïve rats, pretreatment with RAP-103 significantly reduced GFAP mRNA levels in morphine-naïve and morphine-treated rats (P<0.05) as shown in FIG. 8A. For GFAP gene expression in the NAC as shown in FIG. 8A, two-way ANOVA indicated a significant effect of morphine treatment [F(1,23)=7.92, P<0.05]. GFAP mRNA levels in rats treated with morphine were significantly enhanced in comparison with drug-naïve rats (P<0.05), and pretreatment with RAP-103 normalized the augmentation in GFAP gene expression (P<0.05) as shown in FIG. 8A.

For IBA1 gene expression as shown in FIG. 8B in the VTA, two-way ANOVA indicated a significant effect of RAP-103 pretreatment [F(1,27)=8.93, P<0.01]. In comparison with drug-naïve rats, pretreatment with RAP-103 significantly increased IBA1 mRNA levels (P<0.05) as shown in FIG. 8B. In the NAC, two-way ANOVA indicated a significant effect of RAP-103 pretreatment [F(1,24)=12.80, P<0.01]. In rats receiving a combination of RAP-103 and morphine, IBA1 mRNA levels were significantly enhanced in comparison to both drug-naïve rats and rats treated with morphine by itself (P<0.01) as shown in FIG. 8B. In the PFC, significant changes in GFAP or IBA1 were not detected (P>0.05).

Blocking chemokine receptors, for example CCR2, CCR5 and CXCR4, enhances the analgesic effects of opioids in preclinical pain assays. However, little is known about the impacts of chemokine receptor antagonists (CRAs) on adverse effects of opioids. As shown herein chemokine receptor blockade reduces adverse opioid effects, including respiratory depression, physical dependence, and reinforcing efficacy. RAP-103, a small peptide that antagonizes multiple chemokine receptors (e.g., CCR2, CCR5, CCR8, CXCR4), reduced morphine-induced respiratory depression, which is considered to be the proximal cause of death in opioid overdose. RAP-103 also reduced the severity of morphine physical dependence, which was evident from a reduction in naloxone-precipitated withdrawal signs in rats exposed to chronic morphine. In SA assays, RAP-103 reduced heroin acquisition (FR schedule) and reinforcing strength (PR schedule) without affecting sucrose acquisition or reinforcing efficacy, signifying opioid-selective effects of RAP-103 that did not extend to a natural reward. In cellular experiments targeting mesolimbic substrates, abstinence (24 h) from chronic morphine exposure resulted in brain-specific changes in chemokine receptor protein levels, with increased levels (CCR2, CXR4) detected in the VTA and decreased levels detected in the NAC (CCR2, CCR5).

Moreover, morphine abstinence enhanced astrocytic mRNA levels in the NAC. RAP-103 normalized both the morphine-induced dysregulation of chemokine receptor (CCR2, CCR5) and increased astrocyte reactivity in the NAC, thus identifying the NAC as a primary site of RAP-103 action.

In respiratory depression experiments, RAP-103 prevented the reduction in oxygen saturation and hypoxia induced by acute exposure to a submaximal analgesic dose (5 mg/kg) of morphine. Rats treated with RAP-103 by itself had normal oxygen saturation (SpO₂) over the 60-min interval following acute morphine exposure. These results suggest that some chemokine receptors, and their ligands, contribute to the regulation of respiration and response to hypoxia. For example, following high altitude-induced hypoxia in human volunteers or rats exposed to a hypobaric chamber, plasma levels of TNF-α, IL-1β, IL-6, and corticotropin-releasing hormone were all increased. In another study, 1 h after hypoxia, IL-6, IL-9, IL-17A, IL-1β, macrophage inflammatory protein 1-a, granulocyte-macrophage colony stimulating factor, and vascular endothelial growth factor were all increased in the plasma of mice. In the brains of hypoxic neonatal rats, enhancement of CXCR4 expression and astrogliosis occurs but pretreatment with dexamethasone induces neuroprotection and downregulates CXCR4 receptors.

The mechanism by which RAP-103 inhibited morphine-induced hypoxia is unclear, but inhibition of CCL2/CCR2 signaling is a possibility given that RAP-103 inhibits both CCR2 (half maximal inhibitory concentration [IC₅₀=4.2 pM] and CCR5 [IC₅₀=0.18 pM]) in monocyte chemotaxis.

Hypoxia can increase gene transcripts of CCL2 and CCR2 as well as other chemokines, such as MIP-1α, 1β, and ICAM-1, in the carotid body, which is an oxygen-sensing chemoreceptor located in bifurcations of the carotid artery. Hypoxia-induced inflammation in the lungs of rats presents quickly, with enhancement of macrophages and extravasation of albumin in bronchoalveolar lavage detected after 1 h of hypoxia, followed, after 2 h, by increases in mRNA levels of TNF-α, macrophage-inflammatory protein 1β (MIP), and CCL2. CCL2 may be a mediator, released by hypoxic alveolar macrophages, to initiate tissue inflammation following hypoxia. Alveolar hypoxia (exposure of 10% oxygen for 60 min) can increase plasma CCL2 levels (in 5 min) in normal rats but not in alveolar macrophage-deprived rats. Additionally, CCL2 given to rats that were not hypoxic caused degranulation of mast cells as well as inflammation in the mesentery similar to hypoxia, and this inflammation was inhibited by pretreatment with a CCR2 antagonist (RS-102895). Thus, in this example, inhibition of morphine-induced hypoxia by RAP-103 may have been through inhibition of CCL2 binding to CCR2 receptor, thereby preventing CCL2 from initiating alveolar and systemic inflammation against hypoxia. Once the alveolar response to hypoxia was decreased by the CCR2 block, extravasation of albumin and edema were prevented and gas exchange was not affected. Further studies are needed to understand the exact mechanism of how RAP-103 prevents hypoxia and determine if the mechanism is peripheral or central, or both.

In the SA experiments, RAP-103 reduced heroin intake. Under a FR-1 schedule of reinforcement, daily administration of RAP-103 delayed the onset, and reduced the magnitude, of heroin acquisition. In addition, in PR challenge experiments, the reinforcing efficacy of heroin was reduced by RAP-103, suggesting that RAP-103 reduced the motivation to respond for heroin. RAP-103 did not impact responding on the inactive lever, making it unlikely that efficacy was due to sedation, locomotor-deficits, or suppression of non-generalized behaviors. Further, RAP-103 did not affect sucrose intake under either FR or PR conditions, suggesting that RAP-103 efficacy was heroin-specific and did not extend to natural reinforcers.

The mechanisms underlying RAP-103 efficacy are not entirely clear, but prevailing literature, directed toward psychostimulants, implicates dopamine interactions with CCR2 and CCR5 receptors, which can be endogenous targets of RAP-103. CCR2 and CCR5 receptors systems, including endogenous chemokine ligands that activate those receptors, are expressed in cortical, striatal and limbic substrates associated with opioid dependence and are linked to behavioral effects of psychostimulant drugs that reflect addiction. For example, in mice, selective CCR2 receptor antagonists (e.g., INCB 3284 or RS504393) can reduce methamphetamine-induced place conditioning, and CCR2 genetic deletion can reduce locomotor sensitization during repeated methamphetamine exposure. A repeated dosing regimen of methamphetamine that produces CPP in mice also enhances CCL2 mRNA levels in the PFC and NAC and increases mRNA levels of CCL7 (monocyte-chemotactic protein 3), another chemokine ligand that activates CCR2 receptors, in the PFC. For cocaine, locomotor sensitization is not detected in mice lacking CCR2 receptors. Cocaine can also cause an increase in CCL2 expression in microglia of mice that is blocked by CCR2 receptor antagonism or genetic deletion. For the CCR5 system, maraviroc, a CCR5 antagonist, can reduce the rewarding efficacy and locomotor-activating effects of cocaine in rats. Further, CCR5 mRNA levels can be upregulated in both the VTA and NAC following exposure to a dosing paradigm of cocaine that produces CPP. In the context of chemokine-dopamine crosstalk, CCL2 administered into the substantia nigra can increase extracellular dopamine in the dorsal striatum of rats and enhances locomotor activity. Moreover, mice lacking CCR5 receptors can have lower numbers of dopamine neurons in the substantia nigra and reduced levels of extracellular dopamine in the striatum. Because psychostimulant exposure can upregulate elements of mesolimbic CCR2 and CCR5 systems (e.g., CCR5 gene expression in VTA and NAC and CCL2 in the NAC), a similar upregulation of CCR2 and CCR5 systems may have occurred during heroin intake and acquisition in the experiments. Indeed, chronic morphine can promote expression and release of CCL5 from primary cortical astrocytes and from astrocytes in the NAC and VTA of morphine-dependent rats.

Dopamine systems can contribute to the rewarding and reinforcing effects of both opioids and psychostimulants, which provides one explanation for the efficacy of RAP-103 against heroin SA is inhibition of mesolimbic dopamine transmission through dual antagonism of mesolimbic CCR2 and CCR5 receptors. For this interpretation, and in the absence of RAP-103, heroin increased dopamine release in the NAC by inhibiting GABA neurons in the VTA and rostromedial tegmental nucleus that synapse onto dopamine neurons in the VTA. Subsequently, this enhanced mesolimbic dopamine transmission during heroin acquisition promoted downstream recruitment and enhancement of mesolimbic CCR2 and CCR5 systems that augmented the effects of dopamine, thereby further facilitating heroin intake and reinforcement. In the presence of RAP-103, CCR2 and CCR5 receptors were blocked, thereby dampening heroin-induced dopamine transmission and reducing heroin intake. In support of the chemokine-dopamine interpretation, enhanced dopamine D₁ and D₂ receptor activities, which are markers of enhanced dopamine transmission, increase calcium mobilization produced by activation of Gαq-coupled receptors such as CCR5. At the second messenger level, CCR2 activation activates extracellular regulated kinase (ERK). Repeated morphine exposure also causes an increase in ERK activity in the VTA and NAC that reflects activation of reward systems, and psychostimulants produce a similar activation of the ERK response in dopaminergic projection areas, including the NAC. Consequently, crosstalk between chemokines and ERK may facilitate heroin intake. It is also worth mentioning that CCR5 genetic deletion increases expression of monoamine oxidase (MAO), an enzyme that catabolizes dopamine, in the midbrain of mice. Thus, it is possible that RAP-103, through CCR5 antagonism, similar to CCR5 deletion, caused upregulation of MAO that contributed to a reduction in heroin acquisition and reinforcing efficacy.

Daily administration of RAP-103 during chronic morphine exposure reduced development of morphine physical dependence. RAP-103, tested across multiple doses, reduced withdrawal responses precipitated by naloxone, such as wet-dog shakes and eye blinking, but was ineffective against teeth chattering and paw tremors. Given that development of morphine physical dependence, and expression of somatic signs of morphine withdrawal, are multifactorial processes involving diverse neural substrates (e.g. locus coeruleus, hypothalamus, NAC) and activation of different neurotransmitter systems, including dopamine and glutamate, delineation of a specific mechanism of RAP-103 action is challenging. Prior work has established that morphine induces expression of pro-inflammatory cytokines and chemokines in the CNS, particularly in brain regions involved in opioid reward and dependence, including the NAC. Moreover, changes in neuroimmune signaling may play a role in behavioral outcomes following opioid administration.

At the cellular level, the influence of spontaneous withdrawal from chronic morphine exposure on chemokine receptor protein levels in the mesolimbic pathway, and the impact of RAP-103 on morphine-induced changes in chemokine markers was examined. Rats were injected with morphine once daily for 10 d, preceded by an injection of RAP-103 or saline, and brains were extracted 24 h after the last morphine injection. Hence, for this paradigm, the results most likely reflect effects of early morphine abstinence, rather than effects of ongoing chronic morphine exposure. For morphine-abstinent rats, region-specific changes in chemokine receptor protein levels were detected. In the VTA, CCR2 and CXCR4 receptor levels were increased, with a trend toward an increase in CCR5. In contrast, in the NAC, chemokine receptor levels were decreased, with significant deficits detected for CCR2 and CXCR5. In keeping with the chemokine-dopamine crosstalk theme, the reduced chemokine receptor levels in the NAC correlate positively with deficits in extracellular dopamine and dopamine transmission that occur during abstinence from chronic opioid exposure. As noted above for the effects of RAP-103 on heroin intake, chronic and ongoing opioid exposure causes an enhancement of dopamine transmission in the NAC that underlies reinforcing efficacy, and the concurrent upregulation of chemokine receptor expression and signaling (CCR2, CCR5 and CXCR4) that occurred during opioid exposure may indeed augment opioid-induced dopamine transmission and opioid reinforcing efficacy. On the other hand, during abstinence from chronic opioid exposure, which is the time point at which cellular markers were quantified in the experiments, it is conceivable that chemokine, as well as dopamine, systems are downregulated. Indeed, during episodes of abstinence, morphine-dependent animals display reduced levels of extracellular dopamine in the. Furthermore, in morphine-dependent rats, dopamine D₁ and D₂ receptor mRNA levels in the NAC are reduced 1 d after abstinence, which matches the time point in the study, with a gradual return to mRNA levels in morphine-naïve control rats by the second and third days of abstinence.

Although dopamine markers were not measured, similar deficits in D₁ and D₂ receptor levels would be predicted in the paradigm, perhaps due to a morphine-induced hyperdopaminergic state in the NAC that causes receptor saturation during exposure, and into early abstinence, that results in compensatory receptor downregulation due to a decline in extracellular dopamine. Similar to dopamine, CCL5 levels in the NAC are increased during chronic morphine exposure, and CCL2, CCL7 and CCR5 levels in the NAC are increased during repeated psychostimulant exposure. Chemokine receptors, while possibly facilitating a hyperdopaminergic state during morphine exposure, may also become saturated, thereby resulting in compensatory downregulation during morphine abstinence that presents as decreased protein levels. Thus, for the NAC, one interpretation is that receptor levels of CCR2, CCR5, and CXCR4 are highly sensitive to opioid-induced changes in dopamine transmission, with receptor levels increasing during opioid exposure and decreasing during early abstinence. In the case in which RAP-103 is administered during morphine exposure, the chemokine receptor block reduces the normal increase in extracellular dopamine in the NAC. Thus, RAP-103, through chemokine receptor blockade, blunts the magnitude of the hyperdopaminergic state that facilitates opioid reinforcing efficacy and normalizes opioid-induced deficits in chemokine receptor levels by preventing downregulation caused by receptor saturation.

In the VTA, effects of morphine on chemokine receptors were different from the NAC. Early abstinence was associated with enhanced levels of chemokine receptors (CCR2, CXCR4) that were not normalized by RAP-103. The opposing changes in chemokine receptor levels in the NAC and VTA during morphine abstinence may be related to the degree of chemokine crosstalk with dopamine and mu-opioid receptors, respectively. In the NAC, which is a dopaminergic projection area, chemokine receptor levels may be impacted more by opioid-induced changes in dopamine transmission than by direct interactions with mu-opioid receptors. In contrast, in the VTA, chemokine receptor levels may be more directly impacted by the level of mu-opioid receptor activity and whether or not morphine exposure is ongoing or withdrawn. In fact, in the experiments, mu-opioid receptor levels in the VTA were increased 24 h after the last morphine injection, possibly indicating compensatory receptor upregulation to counteract the reduction in dopamine released into the NAC during opioid withdrawal. Interestingly, this mu-opioid receptor upregulation in the VTA during early morphine abstinence was accompanied by upregulation of both CCR2 and CXCR4 receptors. These directionally similar effects of morphine abstinence on mu-opioid, CCR2 and CXCR4 receptor levels may indicate cooperative effects on mesolimbic dopamine output. For example, in the case of CXCR4, a cellular location has not been identified in the VTA, but CXCR4 receptors are expressed by dopamine neurons in the substantia nigra, and intranigral injection of CXCL12 enhances extracellular dopamine levels in the dorsal striatum by activating CXCR4 receptors. Thus, a parallel situation may exist in the VTA whereby CXCR4 receptor activation facilitates mesolimbic dopamine release, and perhaps dopamine synthesis, during morphine exposure. However, during morphine abstinence, when mesolimbic dopamine output declines, tone at CXCR4 receptors may also decrease, leading to a compensatory increase in CXCR4 protein detected in the present experiments. A similar scenario may apply to CCR2, which is expressed on dopaminergic neurons in the substantia nigra, and when activated, enhances dopamine release. Future immunohistochemical studies will identify the types of cells and neurons in the VTA that express CXCR4 and CCR2 to better elucidate mechanism.

Another possibility for the findings in the VTA is that heterologous desensitization, where one type of receptor binds its ligand and the other type of receptor becomes refractory to its ligand, occurred between chemokine and opioid receptors in the VTA. Opioids can desensitize immune cells to movement directed by chemokines, and the inverse process can also occur, as chemokines desensitize mu-opioid receptors located on neurons in the dorsal root ganglion. Given the close association between opioid and chemokine receptors in the VTA, mu-opioid receptor activation during morphine exposure may have desensitized CXCR4 and CCR2 receptors, leading to a compensatory upregulation in chemokine receptor protein levels during exposure, and extending into early abstinence. If so, the inverse is also possible, whereby CXCR4 and CCR2 receptor activation caused desensitization of mu-opioid receptors, leading to compensatory upregulation of mu-opioid receptors in early abstinence. RAP-103, administered during chronic morphine exposure, normalized the deficit in mu-opioid receptor levels detected during morphine abstinence. One explanation for the effects of RAP-103 can be that heterologous desensitization of mu-opioid receptors during chronic morphine exposure was abolished in the presence of the chemokine receptor block by RAP-103, thus preventing the normal upregulation of mu-opioid receptors during abstinence. Conversely, RAP-103 did not affect the enhancement of CXCR4 or CCR2 receptors that occurred during morphine abstinence. The lack of effect is unclear, but one reason may be that RAP-103 blocks activation of CXCR4 and CCR2 without reducing, or normalizing, overall chemokine receptor expression.

Because glial cells release cytokines and chemokines, including those that activate CCR2, CCR5 and CXCR4, changes in astrocyte and microglial reactivity during morphine abstinence, and the impact of RAP-103 on those changes in reactivity were examined. During morphine abstinence, GFAP mRNA levels were increased in comparison to drug-naïve rats, suggesting that astrocyte reactivity was increased 24 h following cessation of chronic morphine exposure. Indeed, dosing regimens of morphine that produce tolerance and dependence can enhance GFAP immunoreactivity in multiple brain regions, including the NAC. In the example, the increase in GFAP mRNA levels during morphine abstinence was specific to the NAC as no significant changes were detected in the VTA or PFC. What is not entirely clear is whether the increase in GFAP mRNA levels, quantified 24 h after discontinuation of morphine exposure, was due to morphine exposure or withdrawal, as previous studies have detected an increase in astrocyte across multiple stages of opioid exposure and withdrawal (e.g., following acute exposure, during chronic exposure, and during abstinence). In keeping with the dopamine-chemokine crosstalk interpretation and evidence that astrocytes are a major source of chemokines, it is possible, in the experiments, that astrocytes in the NAC were activated during chronic opioid exposure and released chemokines, such as CCL2 and CCL5, that facilitated dopamine transmission and opioid reinforcement. The enhanced astrocytic reactivity may have extended into early morphine abstinence to counter the hypodopaminergic state in the NAC by continuing to release chemokines. In support of the interpretation, chronic morphine exposure can increase the expression and release of CCL5 from astrocytes in the NAC of morphine-dependent rats. As was the case for RAP-103 in normalizing morphine-induced dysregulation of chemokine receptors, the NAC may have been the primary site of action here as RAP-103 normalized the increase in GFAP mRNA levels in the NAC detected during morphine abstinence. RAP-103, given throughout chronic morphine exposure, may have disrupted dopamine-chemokine by blocking multiple chemokine receptors, thereby dampening the enhanced dopamine transmission that enhances astrocyte reactivity. Future studies will further explore these astrocytic mechanisms by assessing GFAP mRNA and protein levels across different stages of opioid exposure and abstinence and investigating impacts of other neurotransmitters, such as glutamate.

RAP-103 enhanced microglial mRNA levels in the VTA and NAC in both morphine-naïve and morphine-abstinent rats. The upregulation of microglial gene expression in the mesolimbic pathway by RAP-103 is different in comparison with pain pathways, where RAP-103 reduces spinal microglial activation and monocyte infiltration. Reasons for the region-specific, and opposing, effects of RAP-103 on microglial markers are unclear. Given that RAP-103 antagonizes multiple chemokine receptors (CCR2, CCR5 and CCR8) with different physiological expression patterns and functions, region-specific changes in microglial markers caused by RAP-103 may be most dependent on the specific receptor(s) that exerts the greatest physiological impact in that region. For example, RAP-103 can inhibit microglial chemotaxis and morphological change through a mechanism involving CCR8, a chemokine receptor that has not been studied extensively in brain reward pathways. In the context of the experiments, which focused on brain reward substrates, the upregulation of microglial mRNA levels by RAP-103 may have involved the CCR2 system and potential crosstalk between CCL2/CCR2 and microglia that occurs across different levels of dopamine transmission. Significant induction of CCL2 from human macrophages occurs across different concentrations of dopamine that approximate basal and stimulated conditions in vivo, including those concentrations produced during exposure to psychostimulants and opioids. Thus, one explanation for the results is that a CCR2 block by RAP-103 prevented the CCL2 that is normally released from microglia from activating CCR2, thereby leading to a compensatory upregulation in microglial activity to synthesize more CCL2, and related cytokines and chemokines, to surmount the CCR2 block. Evidence that CCL2 is released from microglia across a broad range of dopamine concentrations, including those approximating levels in the VTA and NAC under basal conditions and during morphine exposure and morphine abstinence, may explain why RAP-103 upregulated microglial mRNA levels in morphine-naïve and morphine-abstinent rats. Because microglial gene expression was not altered in any of the brain regions (VTA, NAC, PFC) in RAP-103 rats receiving only morphine, it is likely that effects on microglial mRNA levels detected in the experiments were primarily due to chemokine receptor blockade by RAP-103.

### Example 2: Synthesis of RAP-103

The synthetic process used for manufacture of RAP-103 Acetate Salt involves the following steps which follow the Merrifield FMOC synthesis method SPPS (Solid phase Peptide Synthesis), using the common commercially available coupling reagents, resins, and deblocking reagents:
Step 1: peptide synthesis: solid phase peptide synthesis (SPPS) of the protected peptide
Step 2: cleavage and deprotection: trifluoroacetic acid (TFA) cleavage of the protecting groups from the peptide and cleavage of the peptide from the resin
Step 3: purification and in-process lyophilization: peptide purification and in-process lyophilization of the peptide
Step 4: ion exchange (salt exchange) and final lyophilization: ion exchange (salt exchange) from TFA to acetate salt and Final lyophilization.

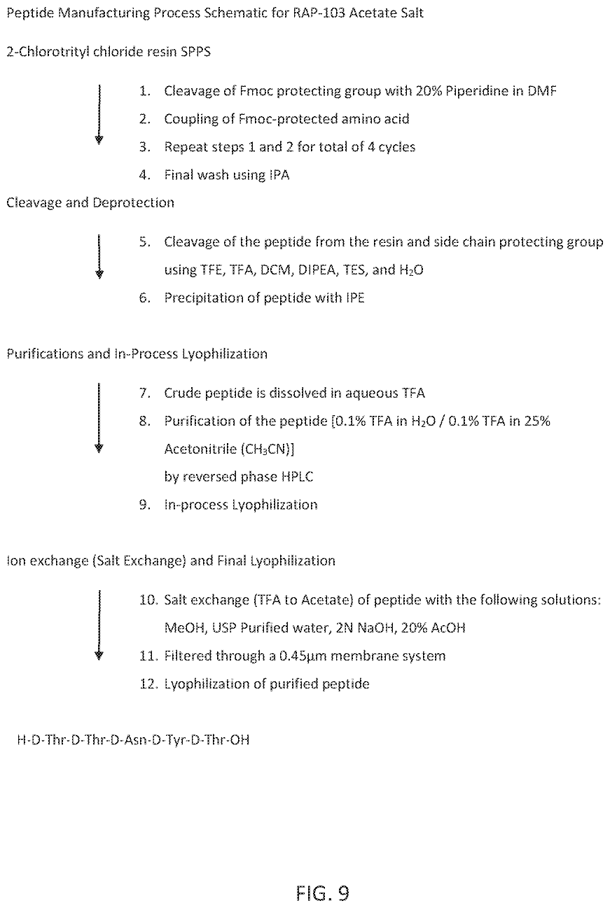

A schematic of a synthesis method for RAP-103 is shown in FIG. 9 and comprises the following steps: 2-chlorotrityl chloride resin SPPS, cleavage and deprotection, purifications and in-process lyophilization, ion exchange (salt exchange) and final lyophilization. The following components are equipment and components were used for synthesis.

### Reaction Vessel

The synthesis was carried out at room temperature in a custom-designed glass vessel, with the bottom part comprising a fritted disk of coarse porosity. The size of the reactor is dependent on the amount of polymer to be used for the synthesis. The reactor was designed to assist in the addition of amino acid derivatives, solvents and reagents, as required. The reaction vessel was equipped with a mechanical stirrer to allow for efficient mixing of the peptide-resin. No components of the equipment or utensils utilized for the synthesis process were composed of materials that can cause adulteration of the product. Solid phase support: 2-Chlorotrityl chloride resin was used for the synthesis.

### Protected Amino Acids

In solid phase peptide synthesis, the reactive functional groups of the amino acids were protected to avoid undesirable side reactions. The protecting groups were of two natures: acid labile and base labile. The base labile protecting group was used to block the α-amino group during the coupling reaction and was removed in the deblocking step, to allow the introduction of the next amino acid in the sequence. Fmoc (9-Fluorenylmethyloxycarbonyl) was used as the base labile a-amino protecting group. The acid labile protecting group was used to protect the side-chain reactive functional groups of the amino acids during synthesis and must be resistant to the deblocking mixture (20% piperidine in DMF). Following the peptide synthesis, these protecting groups were removed by strong acid (aqueous trifluoroacetic acid with scavengers). The acid-labile protecting groups for this process are t-butyl (tBu), trityl (Trt).

The following amino acids were used in the synthesis of RAP-103 Acetate Salt: Fmoc-D-Tyr (tBu)-OH, Fmoc-D-Asn (Trt)-OH, and Fmoc-D-Thr (tBu)-OH.

### Step 1: Peptide Synthesis

Resin Loading-2-Chlorotrityl chloride (CTC) resin was activated with Acetyl chloride (AcCl) and then treated with Fmoc-D-Thr (tBu)-OH and Diisopropylethylamine (DIPEA) followed by a solution mixture of Dichloromethane (DCM), Methanol (MeOH) and Diisopropylethylamine (DIPEA).

The solid phase peptide synthesis by Fmoc strategy can be divided into the following steps:

### Fmoc Deprotection:

During the deprotection step, the base-labile temporary protecting group (Fmoc) was cleaved from the a amino function of the N-terminal amino acid on the growing peptide chain by treating the resin twice with a solution of 20% piperidine in dimethylformamide (DMF). Two deprotection treatments were performed, the 1st deprotection stir time was approximately 10 minutes, and the 2nd deprotection stir time was approximately 30 minutes.

Wash cycle:

The wash steps were performed to eliminate excess reagents used in the preceding step. The solvents selected for each step were carefully chosen to ensure that there is no risk of introducing an undesirable side reaction while eliminating the excess of reagents as efficiently as possible. The duration of each wash step was timed to allow for thorough contact of the peptide-resin with the solvent and to provide ample time for extraction of the reagents. DMF was used after deblocking as well as after coupling because it has excellent solubilizing and swelling properties for all reagents used in the coupling step. Conversely, isopropanol (IPA) was utilized after coupling reaction because it shrinks the resin, which also aids in removal of excess solvents and reagents.

### Activation and Coupling:

During the activation and coupling steps, the deprotected a-amino group is acylated by the next activated amino acid in the sequence. The reagents used to accomplish acylation were carefully selected to create optimal reaction conditions and easy elimination of the excess reagents at the end of the coupling reaction.

Activation of Fmoc-Tyr ((Bu)-OH was performed by dissolving the protected amino acid with coupling reagents 1-H-Benzotriazolium, 1-[bis(dimethylamino)methylene]-5-chloro-tetrafluoroborate (1-),3-oxide (TCTU) and diisopropylethylamine (DIPEA) in DMF. The solution of activated amino acid was then added to the peptide-resin. The mixture was stirred at room temperature for 20 minutes and then DIPEA in DMF was added in it. The mixture was allowed to react for approximately 160 minutes.

Activation of the remaining amino acid derivatives was performed by dissolving the protected amino acid with coupling reagents Oxima (Oxymapure) in DMF and 1,3-Diisopropylcarbodiimide (DIC). The solution of activated amino acid was then added to the peptide-resin. The suspension was stirred at room temperature for 20 minutes, after which a second aliquot of DIC was added to the reaction mixture. The mixture was stirred and allowed to react for approximately 160 minutes.

### Recoupling and Acetylation:

After a minimal reaction time of one hour, the presence of remaining unreacted amino groups was monitored using the qualitative TNBS (trinitrobenzenesulfonic acid) test or the Ninhydrin test. The TNBS test is performed adding a few drops of trinitrobenzenesulfonic acid to the peptide-resin in a test tube sample and allowing the two to react for three minutes. The presence of free amino groups causes a colored reaction; orange-colored beads indicate incomplete coupling and the presence of unreacted amine. Similarly, in the Ninhydrin test, a few drops of the Ninhydrin reagents were added to a sample of the peptide-resin in a small test tube. Blue-stained resin beads indicate the presence of unreacted amine.

If some residual amino groups were detected by either of the above-mentioned tests, the coupling reaction was repeated using half the amount of amino acid derivative required for the first coupling reaction. The Ninhydrin test was performed each time coupling takes place to visualize the presence of unreacted a-amino functions. No recoupling reactions were required during the manufacture of RAP-103 Acetate Salt lot 1000008388.

If unreacted a-amino functions are still present after recoupling, they are acetylated using acetic anhydride to avoid undesirable deletion sequences in the next cycle. No acetylation reactions were required during the manufacture of RAP-103 Acetate Salt lot 1000008388.

After coupling of the last amino acid in the sequence was completed, the peptide-resin was thoroughly washed using Isopropyl Alcohol (IPA) and weighed.

### Step 2: Cleavage and Deprotection

During the cleavage operation, the peptide was detached from the resin with concomitant cleavage of the side chain protecting groups. This was accomplished by the treatment of the peptide-resin with trifluoroacetic acid (TFA) in the presence of scavengers and Trifluoroethanol (TFE) in TFA and Dichloromethane (DCM). Triethylsilane (TES) and water acted as scavengers and were used to provide a protonated cleavage environment which in turn gives higher quality crude. Following the cleavage operation, the peptide was precipitated using cooled isopropyl ether (IPE), and filtered using a Buchner funnel with filter paper. The precipitated peptide was washed with IPE and dried in a vacuum oven at room temperature. After drying was completed, the crude peptide was weighed and recorded.

### Step 3: Purification and in-Process Lyophilization

The purification is performed by preparative Reversed Phase High Performance Chromatography (RP-HPLC).

### Equipment:

The purification equipment was based on the principle of compression in which the chromatographic support is packed in a compression module. A constant pressure was applied to the column. Different column sizes are available and the choice of which to use is based on the amount of material to be processed. The solvents were delivered through pumps and the necessary gradients are created manually or with an automatic gradient maker.

### Nature of Support:

The purification of crude peptide was accomplished by preparative HPLC using reversed phase material as the support. The reversed phase material consists of a silica gel coated with aliphatic chains; the free remaining silanol groups have been end-capped to avoid undesirable ionic interaction/binding between the mixture to be purified and the support. The separation was based on the hydrophobic interaction between the peptide and the resin support. The use of different buffer systems in subsequent purification steps also improves the separation efficiency.

### Purification Using an Aqueous TFA/Acetonitrile Buffer Gradient:

A typical purification run consists of three steps: equilibration Luna C18 column, loading and elution of the product, and washing of the column to prepare it for the next run. Equilibration of the column was accomplished by washing it with aqueous TFA solution. The crude peptide was dissolved in an aqueous TFA solution, filtered and then was loaded onto the column. Product elution was achieved using a gradient of aqueous TFA and acetonitrile (CH3CN) buffer solutions. After product elution, the column was washed with aqueous acetonitrile to check the absence of product. The quality of each different fraction that were collected as the peptide elutes from the column was monitored by analytical HPLC. The fractions, which met the acceptance criteria for purity, were pooled as the main pool and proceed to the next step.

### In-Process Lyophilization:

The main pool of the product from the TFA purification step was filtered through a 0.45 μm membrane filter and lyophilized.

### Step 4: Ion-Exchange (Salt Exchange) and Final Lyophilization

### Ion-Exchange (Salt Exchange):

The Ion-exchange, also referred to as the salt exchange stage, converts the peptide into the required salt form (acetate salt). The preparation of ion-exchange resin (AMBERLITE IRN 78) was accomplished by washing the resin sequentially with methanol (MeOH), USP Water, 2N sodium hydroxide (NaOH), USP water, acetic acid (AcOH-20% in USP water) and USP water until neutrality. The peptide from in-process lyophilization step was dissolved in USP water and loaded onto already prepared Ion-exchange resin. After circulating for two hours, the peptide solution was eluted and collected in fraction collecting bottles. All fractions that met the establish criteria after analyzing with HPLC were collected and pooled together.

### Final Lyophilization

The main pool of the peptide solution from ion-exchange (salt exchange) step was filtered through 0.45 μm membrane filtration cap. The resulting filtrate (peptide solution) was lyophilized to obtain a bulk RAP-103 acetate salt peptide.

### Example 3: Administration of RAP-103

Animals (rats, guinea pigs, and non-human primates (Rhesus macaques; Macaca mulatta) were administered RAP-103 by oral gavage and IV routes. Blood samples were collected into K2EDTA MAP tubes, placed on wet ice, and processed to plasma (in a centrifuge set to maintain 2000 g, at 4 °C for 15 minutes) within 60 minutes of collection and were stored in a freezer set to maintain −80 °C until analysis. Samples were analyzed by using a qualified high performance liquid chromatography (HPLC) with mass spectrometric (MS/MS) detection to determine the concentrations of RAP-103. Giving dosage forms to animals shows that RAP-103 quickly entered the brain (rats and guinea pigs) by IV or oral gavage dosing and persisted at therapeutic levels for at least 24 hrs as shown in FIG. 10A. RAP-103 preferentially enters the brain compared to plasma levels. In non-human primates (rhesus monkeys) RAP-103 was dosed once on Day 1 by intravenous bolus injection at 1 mg/kg and plasma levels determined. The Cmax and AUC 0-T were comparable in male and female monkeys. RAP-103 was highly bioavailable at the 1 mg/kg dose level with absolute bioavailability value of 88% and 89% in females and males. Drug in plasma after a single IV dose (1 mg/Kg) was still measurable at anticipated therapeutic levels 96 hrs. post-dose in non-human primates as shown in FIG. 10B. There were no RAP-103-related changes noted in clinical observations or body weights over seven days indicating no acute toxicity.

### Example 4: Treatment of Opioid-Induced Respiratory Depression with RAP-103

A male subject is prescribed a high level of opioids to treat a pain. The subject is concurrently administered RAP-103 to treat opioid-induced respiratory depression. RAP-103 is orally administered to the subject as a pill to treat the opioid-induced respiratory depression. After administration, the male subject has no change in his respiratory rate and blood oxygen levels (e.g., maintains full respiratory function).

While preferred aspects of the present disclosure have been shown and described herein, it will be obvious to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the methods presented in the disclosure. It is intended that the following claims define the scope of the disclosure and that methods and structures within the scope of these claims and their equivalents be covered thereby.

What is claimed is:

1. A method of treating an opioid-induced respiratory depression in a subject in need thereof, the method comprising administering to the subject a therapeutically effective amount of a pharmaceutical composition to treat the opioid-induced respiratory depression, wherein the pharmaceutical composition comprises:
a peptide, a derivative thereof, or a salt thereof, wherein the peptide comprises at least five contiguous amino acids or derivatives thereof comprising the general formula: E-F-G-H-I, wherein:
E is D-Ser, D-Thr, D-Asn, D-Glu, D-Arg, D-Ile, or D-Leu, or a derivative of any of these;
F is D-Ser, D-Thr, D-Asp, or D-Asn, or a derivative of any of these;
G is D-Thr, D-Ser, D-Asn, D-Arg, D-Gln, D-Lys, or D-Trp, or a derivative of any of these;
His D-Tyr, or a derivative thereof; and
I is D-Thr, D-Ser, D-Arg, or Gly, or a derivative of any of these or a DAPTA, a salt thereof, or a derivative thereof, and an excipient, a diluent, a carrier, or a combination thereof, and wherein the administering of the pharmaceutical composition treats the opioid-induced respiratory depression.

2. The method of claim 1, wherein the peptide, the derivative thereof, or the salt thereof comprises at least eight contiguous amino acids or derivatives thereof, comprising the general formula A-B-C-E-F-G-H-I, and wherein:
A is D-Ala, or a derivative thereof;
B is D-Ser, or D-Thr, or a derivative of any of these;
C is D-Ser, or D-Thr, or a derivative of any of these;
E is D-Ser, D-Thr, D-Asn, D-Glu, D-Arg, D-Ile, or D-Leu, or a derivative of any of these;
F is D-Ser, D-Thr, D-Asp, or D-Asn, or a derivative of any of these;
G is D-Thr, D-Ser, D-Asn, D-Arg, D-Gln, D-Lys, or D-Trp, or a derivative of any of these;
H is D-Tyr, or a derivative thereof; and
I is D-Thr, D-Ser, D-Arg, or Gly or a derivative of any of these.

3. The method of claim 1, wherein the peptide, the derivative thereof, or the salt thereof is D-Thr, D-Thr, D-Asn, D-Tyr, and D-Thr or a salt thereof.

4. The method of claim 1, comprising the derivative of I, wherein I is esterified, glycosylated, or amidated at the C terminus.

5. The method of claim 1, comprising the peptide, the derivative thereof, or the salt thereof, which comprises DAPTA, the salt thereof, or the derivative thereof.

6. The method of claim 1, wherein the pharmaceutical composition is in unit dose form.

7. The method of claim 1, wherein the pharmaceutical composition comprises an excipient.

8. The method of claim 1, wherein prior to the administering the subject was administered an opioid or a salt thereof.

9. The method of claim 1, wherein after or concurrently with the administering, the subject is administered an opioid or a salt thereof.

10. The method of claim 1, wherein after the administering the subject has an increased oxygen saturation level as compared to a subject in need thereof who did not receive the pharmaceutical composition.

11. The method of claim 10, wherein the oxygen saturation level is at least 4%, 5%, 6%, 7%, 8%, 9%, or 10% higher in the subject after the administering as compared to the subject in need thereof who did not receive the pharmaceutical composition as measured by mean percent oxygen saturation.

12. The method of claim 1, wherein after the administering the subject has a reduced respiratory rate as compared to a subject in need thereof who did not receive the pharmaceutical composition.

13. The method of claim 12, wherein the respiratory rate is at least 10%, 15%, 20% or 25% lower in the subject after the administering as compared to the subject in need thereof who did not receive the pharmaceutical composition as measured by cumulative respiratory rate.

14. The method of claim 1, wherein the administering is daily, weekly, or monthly.

15. The method of claim 1, wherein administering is once, twice, three, or four times per day.

16. The method of claim 1, wherein the pharmaceutical composition is administered for about: one day, two days, three days, four days, five days, six days, one week, two weeks, three weeks, four weeks, five weeks, one month, two months, three months, four months, five months, six months, seven months, eight months, nine months, ten months, eleven months, one year, two years, or for life.

17. The method of claim 1, wherein pharmaceutical composition comprises the peptide, the derivative thereof, or the salt thereof in an amount of from about 0.005 mg to about 1000 mg.

18. The method of claim 1, wherein the pharmaceutical composition is administered by: an oral route, an injection route, a sublingual route, a buccal route, a rectal route, a vaginal route, an ocular route, an otic route, a nasal route, an internasal route, an inhalation route, a cutaneous route, a subcutaneous route, an intramuscular route, an intravenous route, a systemic route, a local route, a transdermal route, or any combination thereof.

19. The method of claim 1, wherein the pharmaceutical composition is formulated for oral administration.

20. The method of claim 19, wherein the pharmaceutical composition is in a form of a pill or a liquid.

21. The method of claim 1, wherein a second therapy is administered concurrently or consecutively.

22. The method of claim 21, wherein the second therapy comprises a naloxone, a salt of thereof, or both.

23. The method of claim 21, wherein the pharmaceutical composition further comprises the second therapy.

24. The method of claim 1, wherein the subject was diagnosed with an opioid use disorder prior to the administering.

25. The method of claim 1, wherein the subject was diagnosed with the opioid-induced respiratory depression prior to the administering.

26. The method claim 24 or 25, wherein the diagnoses comprise an in vitro test, a physical exam, an imaging diagnostic or a combination thereof.

27. The method of claim 1, wherein the subject is a mammal.

28. The method of claim 27, wherein the mammal is a human.


SEQUENCE LISTING (23 sequences)

SEQ ID NO: 1 - protein, 5 aa, synthetic construct
  SITE 1: note = D-amino acid
  SITE 2: note = D-amino acid
  SITE 3: note = D-amino acid
  SITE 4: note = D-amino acid
  SITE 5: note = D-amino acid
TTNYT

SEQ ID NO: 2 - protein, 5 aa, synthetic construct
  SITE 1: note = D-amino acid
  SITE 2: note = D-amino acid
  SITE 3: note = D-amino acid
  SITE 4: note = D-amino acid
  SITE 5: note = D-amino acid
SSTYR

SEQ ID NO: 3 - protein, 5 aa, synthetic construct
  SITE 1: note = D-amino acid
  SITE 2: note = D-amino acid
  SITE 3: note = D-amino acid
  SITE 4: note = D-amino acid
  SITE 5: note = D-amino acid
STNYT

SEQ ID NO: 4 - protein, 5 aa, synthetic construct
  SITE 1: note = D-amino acid
  SITE 2: note = D-amino acid
  SITE 3: note = D-amino acid
  SITE 4: note = D-amino acid
NTSYG

SEQ ID NO: 5 - protein, 8 aa, synthetic construct
  SITE 1: note = D-amino acid
  SITE 8: note = Amidated residue
ASTTTNYT

SEQ ID NO: 6 - protein, 8 aa, synthetic construct
  SITE 1: note = D-amino acid
ASTTTNYT

SEQ ID NO: 7 - protein, 8 aa, synthetic construct
  SITE 1: note = D-amino acid
  SITE 2: note = D-amino acid
  SITE 3: note = D-amino acid
  SITE 4: note = D-amino acid
  SITE 5: note = D-amino acid
  SITE 6: note = D-amino acid
  SITE 7: note = D-amino acid
  SITE 8: note = Amidated D-amino acid
ASTTTNYT

SEQ ID NO: 8 - protein, 8 aa, synthetic construct
  SITE 1: note = D-amino acid
  SITE 2: note = D-amino acid
  SITE 3: note = D-amino acid
  SITE 4: note = D-amino acid
  SITE 5: note = D-amino acid
  SITE 6: note = D-amino acid
  SITE 7: note = D-amino acid
  SITE 8: note = D-amino acid
ASTTTNYT

SEQ ID NO: 9 - protein, 5 aa, synthetic construct
  SITE 1: note = D-amino acid
  SITE 2: note = D-amino acid
  SITE 3: note = D-amino acid
  SITE 4: note = D-amino acid
  SITE 5: note = D-amino acid
ETWYS

SEQ ID NO: 10 - protein, 5 aa, synthetic construct
  SITE 1: note = N-methylated D-amino acid
  SITE 2: note = D-amino acid
  SITE 3: note = D-amino acid
  SITE 4: note = D-amino acid
  SITE 5: note = D-amino acid
TTNYT

SEQ ID NO: 11 - protein, 5 aa, synthetic construct
TTNYT

SEQ ID NO: 12 - protein, 5 aa, synthetic construct
SSTYR

SEQ ID NO: 13 - protein, 5 aa, synthetic construct
STNYT

SEQ ID NO: 14 - protein, 8 aa, synthetic construct
ASTTTNYT

SEQ ID NO: 15 - protein, 8 aa, synthetic construct
  VARIANT 1: note = D or L-amino acid
  VARIANT 2: note = D or L-amino acid
  VARIANT 3: note = D or L-amino acid
  VARIANT 4: note = D or L-amino acid
  VARIANT 5: note = D or L-amino acid
  VARIANT 6: note = D or L-amino acid
  VARIANT 7: note = D or L-amino acid
  VARIANT 8: note = D or L-amino acid
ASTTTNYT

SEQ ID NO: 16 - protein, 5 aa, synthetic construct
  VARIANT 1: note = D or L-amino acid
  VARIANT 2: note = D or L-amino acid
  VARIANT 3: note = D or L-amino acid
  VARIANT 4: note = D or L-amino acid
  VARIANT 5: note = D or L-amino acid
SSTYR

SEQ ID NO: 17 - protein, 5 aa, synthetic construct
  VARIANT 1: note = D or L-amino acid
  VARIANT 2: note = D or L-amino acid
  VARIANT 3: note = D or L-amino acid
  VARIANT 4: note = D or L-amino acid
  VARIANT 5: note = D or L-amino acid
STNYT

SEQ ID NO: 18 - protein, 5 aa, synthetic construct
  VARIANT 1: note = D or L-amino acid
  VARIANT 2: note = D or L-amino acid
  VARIANT 3: note = D or L-amino acid
  VARIANT 4: note = D or L-amino acid
NTSYG

SEQ ID NO: 19 - protein, 5 aa, synthetic construct
NTSYG

SEQ ID NO: 20 - protein, 5 aa, synthetic construct
  VARIANT 1: note = D or L-amino acid
  VARIANT 2: note = D or L-amino acid
  VARIANT 3: note = D or L-amino acid
  VARIANT 4: note = D or L-amino acid
  VARIANT 5: note = D or L-amino acid
TTNYT

SEQ ID NO: 21 - protein, 8 aa, synthetic construct
  VARIANT 1: note = D or L-amino acid
  VARIANT 2: note = D or L-amino acid
  VARIANT 3: note = D or L-amino acid
  VARIANT 4: note = D or L-amino acid
  VARIANT 5: note = D or L-amino acid
  VARIANT 6: note = D or L-amino acid
  VARIANT 7: note = D or L-amino acid
  VARIANT 8: note = Amidated D or L-amino acid
ASTTTNYT

SEQ ID NO: 22 - protein, 8 aa, synthetic construct
  SITE 8: note = Amidated residue
ASTTTNYT

SEQ ID NO: 23 - protein, 8 aa, synthetic construct
  SITE 1: note = D-amino acid
  SITE 2: note = D-amino acid
  SITE 3: note = D-amino acid
  SITE 4: note = D-amino acid
  SITE 5: note = D-amino acid
  SITE 6: note = D-amino acid
  SITE 7: note = D-amino acid
  SITE 8: note = Amidated D-amino acid
ASTTTNYT